(19) United States
(12) Patent Application Publication
(10) Pub. No.: US 2026/0283498 A1
(43) Pub. Date: Sep. 24, 2026

(54) METHODS, SYSTEMS, DEVICES, AND STORAGE MEDIUM FOR MAGNETIC RESONANCE IMAGING

(71) Applicant: SHANGHAI UNITED IMAGING HEALTHCARE CO., LTD., Shanghai (CN)

(72) Inventors: Xiangbin FANG, Shanghai (CN); Can LIAO, Shanghai (CN)

(73) Assignee: SHANGHAI UNITED IMAGING HEALTHCARE CO., LTD., Shanghai (CN)

(21) Appl. No.: 19/466,493

(22) Filed: Feb. 1, 2026

Related U.S. Application Data
(63) Continuation of application No. PCT/CN2024/114388, filed on Aug. 24, 2024.

(30) Foreign Application Priority Data
Sep. 1, 2023 (CN) 202311127257.9

(51) Int. Cl. A61B 5/055; A61B 5/00; G06T 7/246

(52) U.S. Cl. CPC A61B 5/055; A61B 5/0037; A61B 5/7214; G06T 7/246; G06T 2207/10088; G06T 2207/20084

(57) ABSTRACT

Embodiments of the present disclosure provide methods, systems, devices, and storage mediums for determining a target motion state and obtaining a target image. The method may include: obtaining first scanning data of a subject during a first scanning stage, the first scanning data corresponding to a first field of view (FOV); obtaining second scanning data of the subject during the first scanning stage, the second scanning data corresponding to a second FOV, the second FOV being smaller than the first FOV; determining, based on the first scanning data and the second scanning data, a target correspondence between the first scanning data and the second scanning data of the subject in at least one motion state during the first scanning stage; and determining, based on the target correspondence, a target motion state of the subject during a second scanning stage.

### CROSS-REFERENCE RELATED TO APPLICATIONS

This application is a continuation of International Application No. PCT/CN2024/114388, filed on 24 Aug. 2024, which claims priority to Chinese Patent Application No. 202311127257.9, filed on Sep. 1, 2023, the entire contents of which are incorporated herein by reference.

### TECHNICAL FIELD

The present disclosure relates to the field of medical imaging, and in particular, to methods, systems, devices, and storage mediums for determining a target motion state and obtaining a target image.

### BACKGROUND

Magnetic resonance imaging (MRI) is widely used in the imaging process of parts such as the liver, the brain, etc., due to its excellent soft tissue imaging ability and safety features of no ionizing radiation. Since the speed of magnetic resonance imaging is limited by factors such as phase encoding and repetition time (TR), imaging of a certain motion organ is often accompanied by motion artifacts. During MRI-guided radiation therapy, a three-dimensional (3D) magnetic resonance scan is required. However, a conventional 3D scanning sequence is unable to meet the requirement of real-time MRI imaging.

In the case of relatively high real-time requirement, the real-time motion state capture of a certain organ that is greatly affected by tissue motion such as respiratory motion, heart beating, etc., may be needed to be achieved. Therefore, it is desirable to provide methods, systems, devices, and storage medium for determining a target motion state rapidly and efficiently, thereby finally realizing high-speed and real-time three-dimensional MRI.

### SUMMARY

One or more embodiments of the present disclosure provide a method for determining a target motion state based on K-space data difference caused by an image wrap phenomena within a small field of view (FOV). The method for determining a target motion state can effectively reduce time consumption of phase encoding and image reconstruction, reduce computing power consumption brought by signal processing and image reconstruction to a computer, and quickly locate a current motion state of the tissue by obtaining high real-time signals of the moving tissue, and finally realize high-speed and real-time three-dimensional imaging of magnetic resonance.

The method may include obtaining first scanning data of a subject during a first scanning stage, and the first scanning data may correspond to a first FOV. The method may include obtaining second scanning data of the subject during the first scanning stage, and the second scanning data may correspond to a second FOV, and the second FOV may be smaller than the first FOV. The method may also include determining, based on the first scanning data and the second scanning data, a target correspondence between the first scanning data and the second scanning data of the subject in at least one motion state during the first scanning stage. The method may further include determining, based on the target correspondence, a target motion state of the subject during a second scanning stage.

The method may include obtaining first scanning data of a subject during a first scanning stage, and the first scanning data may correspond to a first FOV. The method may include obtaining second scanning data of the subject during the first scanning stage, the second scanning data may correspond to a second FOV, and the second FOV may be smaller than the first FOV. The method may include determining, based on the first scanning data and the second scanning data, a target correspondence between the first scanning data and the second scanning data of the subject in at least one motion state during the first scanning stage. The method may further include determining, based on the target correspondence, a target image of the subject during a second scanning stage.

One or more embodiments of the present disclosure provide a device. The device may include at least one processor and at least one storage. The at least one storage may be configured to store computer instructions. The at least one processor may be configured to execute at least a portion of the computer instructions to obtain first scanning data of a subject during a first scanning stage, the first scanning data corresponding to a first FOV, obtain second scanning data of the subject during the first scanning stage, the second scanning data corresponding to a second FOV, and the second FOV being smaller than the first FOV, determine, based on the first scanning data and the second scanning data, a target correspondence between the first scanning data and the second scanning data of the subject in at least one motion state during the first scanning stage, and determine, based on the target correspondence, a target motion state of the subject during a second scanning stage.

One or more embodiments of the present disclosure provide a device. The device may include at least one processor and at least one storage. The at least one storage may be configured to store computer instructions. The at least one processor may be configured to execute at least a portion of the computer instructions to obtain first scanning data of a subject during a first scanning stage, the first scanning data corresponding to a first FOV, obtain second scanning data of the subject during the first scanning stage, the second scanning data corresponding to a second FOV, and the second FOV being smaller than the first FOV, determine, based on the first scanning data and the second scanning data, a target correspondence between the first scanning data and the second scanning data of the subject in at least one motion state during the first scanning stage, and determine, based on the target correspondence, a target image of the subject during a second scanning stage.

One or more embodiments of the present disclosure provide a computer-readable storage medium storing computer instructions. When reading the computer instructions in the storage medium, the computer may perform a method. The method may include obtaining first scanning data of a subject during a first scanning stage, and the first scanning data may correspond to a first FOV. The method may include obtaining second scanning data of the subject during the first scanning stage, the second scanning data may correspond to a second FOV, and the second FOV may be smaller than the first FOV. The method may also include determining, based on the first scanning data and the second scanning data, a target correspondence between the first scanning data and the second scanning data of the subject in at least one motion state during the first scanning stage. The method may further include determining, based on the target correspondence, a target motion state of the subject during a second scanning stage.

One or more embodiments of the present disclosure provide a computer-readable storage medium storing computer instructions. When reading the computer instructions in the storage medium, the computer may perform a method. The method may include obtaining first scanning data of a subject during a first scanning stage, the first scanning data may correspond to a first FOV, and the first scanning data may include at least one combination of 3D images of the subject in at least one motion state during the first scanning stage. The method may include obtaining second scanning data of the subject during the first scanning stage, the second FOV may correspond to a second FOV, and the second FOV may be smaller than the first FOV. The method may include determining, based on the first scanning data and the second scanning data, a target correspondence between the first scanning data and the second scanning data of the subject in at least one motion state during the first scanning stage. The method may include obtaining third scanning data of the subject during the second scanning stage. The method may further include determining, based on the target correspondence, a target image of the subject during a second scanning stage.

One or more embodiments of the present disclosure provide a method. The method may include obtaining first scanning data of a subject during a first scanning stage, and the first scanning data may correspond to a first FOV. The method may include obtaining second scanning data of the subject during the first scanning stage, the second scanning data may correspond to a second FOV, and the second FOV may be smaller than the first FOV. The method may include determining, based on the first scanning data and the second scanning data, a target correspondence between the first scanning data and the second scanning data of the subject in at least one motion state during the first scanning stage. The method may include determining, based on the target correspondence, a target image of the subject during a second scanning stage. The method may further include determining, based on the target image, a radiotherapy plan of the subject.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a diagram illustrating an exemplary system for magnetic resonance imaging (MRI) according to some embodiments of the present disclosure;

FIG. 2 is a schematic diagram illustrating an exemplary system for MRI according to some embodiments of the present disclosure;

FIG. 3 is a flowchart illustrating an exemplary process for determining a target motion state according to some embodiments of the present disclosure;

FIG. 4 is a schematic diagram illustrating obtaining a three-dimensional scanning image by performing a three-dimensional state segmentation according to some embodiments of the present disclosure;

FIG. 5 is a schematic diagram illustrating an exemplary relationship between a second field of view (FOV) and a subject according to some embodiments of the present disclosure;

FIG. 6 is an exemplary schematic diagram illustrating dividing a K-space dataset using a K-space data variation feature according to some embodiments of the present disclosure;

FIG. 7 is a schematic diagram illustrating an exemplary relationship between image wrap and K-space datasets according to some embodiments of the present disclosure;

FIG. 8 is an exemplary schematic diagram illustrating obtaining a target correspondence according to some embodiments of the present disclosure;

FIG. 9 is a schematic diagram illustrating an exemplary for determining a target motion state according to some embodiments of the present disclosure;

FIG. 10 is a flowchart illustrating an exemplary process for obtaining a target image according to some embodiments of the present disclosure; and

FIG. 11 is a flowchart illustrating an exemplary process determining a radiotherapy plan according to some embodiments of the present disclosure.

### DETAILED DESCRIPTION

In order to more clearly illustrate the technical solutions relating to the embodiments of the present disclosure, a brief introduction of the drawings referred to the description of the embodiments is provided below. Obviously, the drawings described below are only some examples or embodiments of the present disclosure. Those having ordinary skills in the art, without further creative efforts, may apply the present disclosure to other similar scenarios according to these drawings. Unless obviously obtained from the context or the context illustrates otherwise, the same numeral in the drawings refers to the same structure or operation.

It should be understood that the “system,” “device,” “unit,” and/or “module” used herein are one method to distinguish different components, elements, parts, sections, or assemblies of different levels. However, if other words can achieve the same purpose, the words can be replaced by other expressions.

As used in the disclosure and the appended claims, the singular forms “a,” “an,” and “the” include plural referents unless the content clearly dictates otherwise; the plural forms may be intended to include singular forms as well. In general, the terms “comprise,” “comprises,” and/or “comprising,” “include,” “includes,” and/or “including,” merely prompt to include steps and elements that have been clearly identified, and these steps and elements do not constitute an exclusive listing. The methods or devices may also include other steps or elements.

The flowcharts used in the present disclosure illustrate operations that the system implements according to the embodiment of the present disclosure. It should be understood that the foregoing or following operations may not necessarily be performed exactly in order. Instead, the operations may be processed in reverse order or simultaneously. Besides, one or more other operations may be added to these processes, or one or more operations may be removed from these processes.

In order to achieve real-time motion state capture of a certain organ that is greatly affected by tissue motion such as respiratory motion, heart beating, etc., and to improve the precision of motion tracking of the target organ and/or tissue, the current motion state of tissue may be usually located by capturing two-dimensional (2D) images. However, the manner has the problem that the speed of 2D magnetic resonance imaging limits the frame rate of real-time imaging. In addition, in 2D imaging, some high-speed imaging sequences (e.g., the fastest planar echo imaging technique available) can be combined with different sequences to achieve fast imaging results. However, since the technique requires a series of gradient alternations to achieve continuous signal acquisition, the technique has an extremely high requirement for gradient performance, and the gradient switching rate is required to be extremely accurate. Due to the loss of phase correction of the nuclei by the recombination pulses, the accumulated phase error leads to more severe image distortion and relevant artifacts. As a result, it is difficult for the technique to meet the requirement of real-time imaging over a long period of time, and the stability of image quality, the gradient performance, and the risk of loss of superconductor will raise the technical threshold.

The parallel MRI imaging technique is the widely used in speeding up imaging. The MRI parallel imaging technique assists in localizing the spatial location of the MR signal using coil information with known location and sensitivity parameters. Therefore, a count of phase encoding steps is reduced, thereby reducing the imaging time. But the simultaneous obtaining of magnetic resonance signals through multi-channel coils may be constrained by limitations of hardware performance such as system memory, and the computing and reconstruction under multi-channel may also bring computational pressure to the system. In small-area local imaging, the limitation of the single-channel coil size makes the parallel imaging of multiple sampling channels, difficult to achieve good results in small-area local imaging.

The compressed sensing technique may be used in speeding up imaging. The compressed sensing technique compensates for the high-frequency position information of under-sampling in the K-space by adopting the conjugate feature of data in K-space and taking the way of down-sampling based on the post-computation of kernel data of central full-sampling, which achieves imaging acceleration through reducing the count of encoding and sampling times. However, the complex reconstruction algorithms and the demand for training data also hinder the realization of the technique.

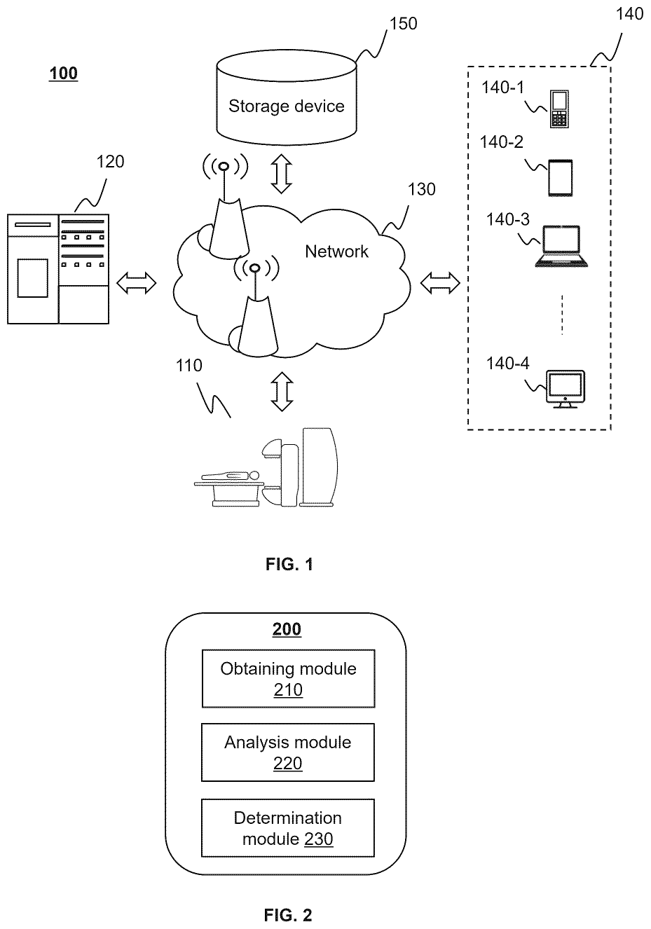

FIG. 1 is a diagram illustrating an exemplary system for magnetic resonance imaging (MRI) according to some embodiments of the present disclosure. As shown in FIG. 1, the system for magnetic resonance imaging (MRI) 100 (also referred to as MRI system) may include a magnetic resonance imaging (MRI) device 110, a processing device 120, a network 130, a terminal device 140, and a storage device 150. In some embodiments, components of the application scenario 100 may be connected to each other via the network 130. In some embodiments, some of the components of the application scenario 100 may be directly connected to each other.

The MRI device 110 may be used to perform a magnetic resonance scan on a subject. In some embodiments, the subject may be biological or non-biological. For example, the subject may include a patient, an artificial object, etc. In some embodiments, the subject may include a specific part of the body, for example, the head, the neck, the chest, or the like, or any combination thereof. In some embodiments, the subject may include a specific organ, for example, a liver, a kidney, the pancreas, the bladder, the uterus, the rectum, or the like, or any combination thereof. In some embodiments, the subject may include a region of interest (ROI), e.g., a tumor, a nodule, etc. In some embodiments, the MRI device may include a multimodal device, such as an MR-radiotherapy device, an MR-positron emission computed tomography (PET) device, etc.

In some embodiments, the MRI device 110 may obtain raw data (e.g., K-space data, etc.) by performing an MR scan on the subject. In some embodiments, the MRI device 110 may obtain image data (e.g., two dimensional images, three dimensional images, four dimensional images) by performing the MR scan on the subject. In some embodiments, the MRI device 110 may obtain three-dimensional scanning data (e.g., a three-dimensional scanning image, etc.) by performing a three-dimensional magnetic resonance scan on the subject. In some embodiments, the MRI device 110 may obtain four-dimensional (4D) scanning data (e.g., a three-dimensional scanning image including temporal dimension information, etc.) by performing a 4D magnetic resonance scan on the subject.

In some embodiments, the MRI device 110 may perform the magnetic resonance scan on the subject according to one or more MR pulse sequences. The MR pulse sequences may include a free induction decay (FID) sequence, a spin echo (SE) sequence, a gradient recalled echo (GRE), or the like. In some embodiments, different MR pulse sequences may be used to scan different subjects. For example, a scan may be performed on the head using the MR pulse sequence of Ax SE T1, and a scan may be performed on the pituitary gland using the MR pulse sequence of Cor SE T1.

In some embodiments, the MRI device 110 may include a 1.5 T magnetic resonance device, a 3.0 T magnetic resonance device, a 5.0 T magnetic resonance device, a 7.0 T magnetic resonance device, etc.

The descriptions regarding the MRI device 110 are merely provided for the purpose of illustration, and not intended to limit the scope of the present disclosure.

In some embodiments, the MRI device 110 may send the scanning data to the processing device 120 and/or other components of the MRI system 100. In some embodiments, the MRI device 110 may perform the magnetic resonance scan by receiving relevant data or instructions from the processing device 120 and/or other components of the MRI system 100.

The processing device 120 may process data and/or information obtained from the MRI device 110, the terminal device 140, and/or the storage device 150. For example, the processing device 120 may obtain first scanning data and second scanning data of the subject during a first scanning stage. The first scanning data may be acquired in a first field of view (FOV) and the second scanning data may be acquired in a second FOV. The first FOV may include the second FOV. The first FOV may exceed the second FOV. The processing device 120 may determine, based on the first scanning data and the second scanning data, a target correspondence between the first scanning data and the second scanning data of the subject in each motion state during a first scanning stage. As another example, the processing device 120 may obtain third scanning data of the subject during a second scanning stage, and determine, based on the third scanning data and the target correspondence, a target motion state of the subject when the third scanning data is acquired by the MRI device and/or a target image of the subject corresponding to the third scanning data.

In some embodiments, the first scanning stage refers to a phase where the target correspondence between the first scanning data and the second scanning data is obtained. The first scanning stage may also be referred to a preprocessing phase or a pre-scanning phase. The second scanning stage refers to a phase where the motion state of the subject second scanning stage is determined according to the target correspondence in real time. The second scanning stage may also be referred to the application phase of the correspondence between the first scanning data acquired under the first FOV and the second scanning data acquired under the second FOV. In some embodiments, motion-related data of the subject during the second scanning stage may be obtained by various means such as a sensor, an imaging device (e.g., a MRI device), etc., and the target motion state of the subject during the second scanning stage (and optionally, other motion cycles similar to the motion cycle corresponding to the scanning process) may be determined based on the motion-related data of the subject combined with the target correspondence.

In some embodiments, the processing device 120 may be a single server or a server group. The server group may be centralized or distributed. In some embodiments, the processing device 120 may be local or remote. For example, the processing device 120 may access information and/or data from the MRI device 110, the terminal device 140, and/or the storage device 150 via the network 130. As another example, the processing device 120 may be directly connected to the MRI device 110, the terminal device 140, and/or the storage device 150 to access the information and/or data. In some embodiments, the processing device 12 may be provided in the MRI device 110. In some embodiments, the processing device 120 may be implemented on a cloud platform. For example, the cloud platform may include a private cloud, a public cloud, a hybrid cloud, a community cloud, a distributed cloud, an inter-cloud, a multi-cloud, or the like, or any combination thereof.

The network 130 may include any suitable network that can facilitate exchange of information and/or data. In some embodiments, the information and/or data may be exchanged between one or more components of the application scenario 100 via the network 130. For example, the processing device 120 may receive scanning data from the MRI device 110 via the network 130. The network 130 may include a local area network (LAN), a wide area network (WAN), a wired network, a wireless network, or the like, or any combination thereof.

The terminal device 140 may communicate with and/or be connected to the MRI device 110, the processing device 120, and/or the storage device 150. For example, the terminal device 140 may send one or more control instructions to the MRI device 110 via the network 130 to control the MRI device 110 to perform a magnetic resonance scan on the subject according to the instructions. In some embodiments, the terminal device 140 may include other devices that have input/output functions such as a mobile device 140-1, a tablet computer 140-2, a laptop computer 140-3, a desktop computer 140-4, or the like, or any combination thereof. In some embodiments, the terminal device 140 may include an input device, an output device, etc. The input device may include a keyboard, a touch screen, a mouse, a voice device, or the like, or any combination thereof. The output device may include a monitor, a speaker, a printer, or the like, or any combination thereof. In some embodiments, the terminal device 140 may be a part of the processing device 120. In some embodiments, the terminal device 140 may be integrated with the processing device 120 as an operating station of the MRI device 110.

The storage device 150 may store data, instructions, and/or any other information. In some embodiments, the storage device 150 may store data obtained from the MRI device 110, the processing device 120, and/or the terminal device 140. For example, the storage device 150 may store raw data obtained from the MRI device 110. In some embodiments, the storage device 150 may store data and/or instructions that the processing device 120 may execute or use to perform exemplary methods described in the present disclosure. For example, the storage device 150 may store instructions for controlling the MRI device 110 to perform a scan.

In some embodiments, the storage device 150 may include a mass storage device, a removable storage device, a volatile read-and-write memory, a read-only memory (ROM), or the like, or any combination thereof. In some embodiments, the storage device 150 may be implemented on a cloud platform. In some embodiments, the storage device 150 may be a part of the processing device 120.

It should be noted that the descriptions are merely provided for the purpose of illustration, and not intended to limit the scope of the present disclosure. For those skilled in the art, various variations and modifications may be made under the teachings of the present disclosure. The features, structures, methods, and other characteristics of the exemplary embodiments described herein may be combined in various ways to obtain additional and/or alternative exemplary embodiments. For example, the MRI device 110, the processing device 120, and the terminal device 140 may share one storage device 150, or may have their own storage devices. However, these variations and modifications do not depart from the scope of the present disclosure.

FIG. 2 is a schematic diagram illustrating an exemplary system for MRI according to some embodiments of the present disclosure. In some embodiments, the MRI system 200 may include an obtaining module 210, an analysis module 220, and a determination module 230. In some embodiments, the MRI system 200 may be implemented by the processing device 120.

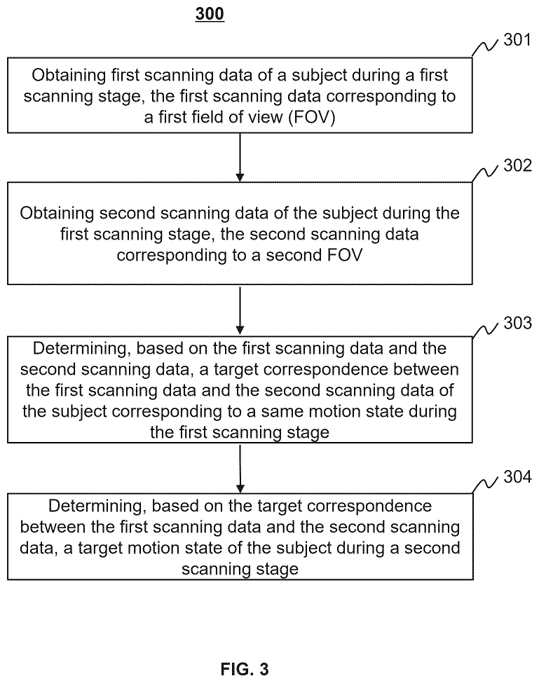

In some embodiments, the obtaining module 210 may be configured to obtain first scanning data of a subject during a first scanning stage, the first scanning data corresponding to a first FOV, and obtain second scanning data of the subject during the first scanning stage, the second scanning data corresponding to a second FOV. More descriptions regarding the obtaining first scanning data and second scanning data may be found in FIG. 3 and the relevant descriptions thereof.

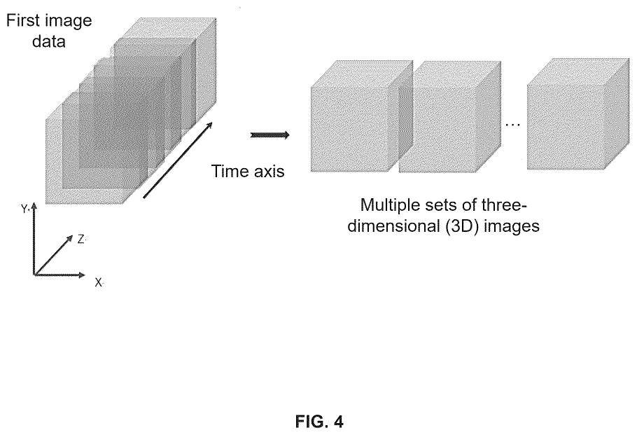

In some embodiments, the first scanning data may include multiple sets of 3D images of the subject during the first scanning stage. Accordingly, the obtaining module 210 may be further configured to obtain first image data of the subject during the first scanning stage, the first image data corresponding to the first FOV, and obtain the multiple sets of first images by performing three-dimensional state segmentation based on the first image data, each set of the multiple sets of 3D images corresponding to one of the at least one motion state of the subject. More descriptions regarding the obtaining the multiple sets of 3D images may be found in FIG. 3, FIG. 4, and the relevant descriptions thereof.

In some embodiments, the second scanning data may include at least one K-space dataset in at least one motion state of the subject during the first scanning stage. Accordingly, the obtaining module 210 may be further configured to obtain the second raw data (i.e., second k-space data) of the subject, the second raw data corresponding to the second FOV, and obtain the at least one K-space dataset by dividing the second raw data based on a K-space data variation feature, each of the at least one K-space dataset corresponding to one of the at least one set of 3D images. More descriptions regarding the obtaining the at least one K-space dataset may be found in FIG. 3 and the relevant descriptions thereof.

In some embodiments, the analysis module 220 may be configured to determine, based on the first scanning data and the second scanning data, a target correspondence between the first scanning data and the second scanning data of the subject corresponding to a same motion state during the first scanning stage. In some embodiments, the analysis module 220 may be further configured to determine, based on the at least one set of 3D images and the at least one K-space dataset, the target correspondence between the K-space datasets of the subject in the at least one motion state during the first scanning stage and the set of 3D images through a matching model. More descriptions regarding the determining the target correspondence may be found in FIG. 3 and the relevant descriptions thereof.

In some embodiments, the determination module 230 may be configured to determine, based on the target correspondence between the first scanning data and the second scanning data, a target motion state of the subject during a second scanning stage. More descriptions regarding the determining a target motion state of a subject during the second scanning stage may be found in FIG. 3 and the relevant descriptions thereof.

In some embodiments, the determination module 230 may be further configured to obtain third scanning data of the subject during the second scanning stage, the third scanning data corresponding to the second FOV, and determine, based on the third scanning data and the target correspondence, a target motion state of the subject when the third scanning data is acquired by an MRI device. More descriptions regarding the third scanning data and the determining a target motion state of the subject when the third scanning data is acquired by an MRI device, may be found in FIG. 3 and the relevant descriptions thereof.

In some embodiments, the determination module 230 may be further configured to determine, based on the third scanning data, a target K-space dataset in the second scanning data matching the third scanning data, and determine, based on the target correspondence and the target K-space dataset matching the third scanning data, a target motion state corresponding to the third scanning data. More descriptions regarding the determining a target motion state may be found in FIG. 3 and the relevant descriptions thereof.

In some embodiments, the determination module 230 may be further configured to determine, based on the target correspondence between the first scanning data and the second scanning data, a target image of the subject during the second scanning stage.

In some embodiments, the determination module 230 may be further configured to obtain third scanning data of the subject during the second scanning stage and determine, based on the third scanning data and the target correspondence, the target image of the subject during the second scanning stage. In some embodiments, the determination module 230 may be further configured to determine, based on the third scanning data, a target K-space dataset in the second scanning data matching the third scanning data and determine the target image corresponding to the third scanning data based on the target correspondence and the target K-space dataset matching the third scanning data. In some embodiments, the determination module 230 may be further configured to determine a matching degree between each K-space dataset in the second scanning data and the third scanning data and determine, based on the matching degree, the target K-space dataset in the second scanning data matching the third scanning data.

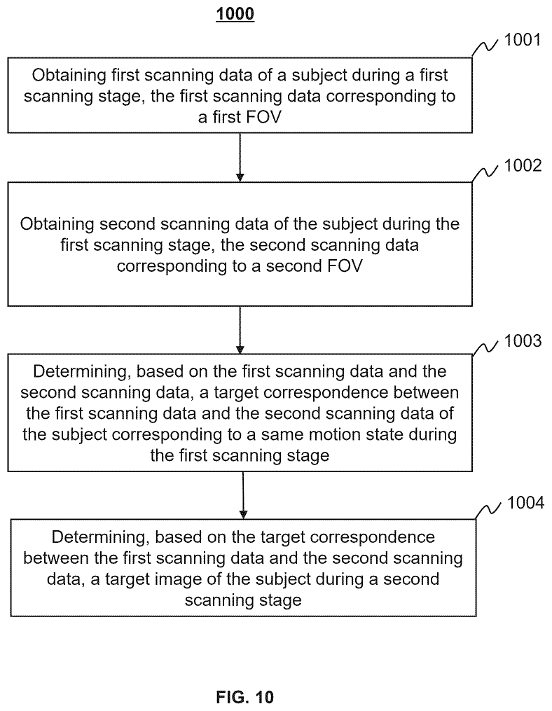

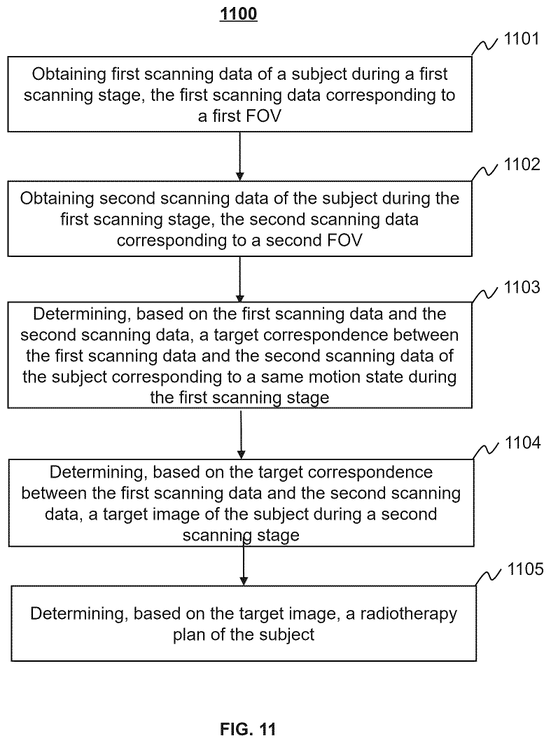

More descriptions regarding the determining the target image during the second scanning stage may be found in FIG. 10 and the relevant descriptions thereof.

In some embodiments, the determination module 230 may be further configured to determine, based on the target image during the second scanning stage, a radiotherapy plan of the subject. More descriptions regarding the determining a radiotherapy plan of the subject may be found in FIG. 11 and the relevant descriptions thereof.

It should be noted that the descriptions of the system 200 for determining a target motion state and the modules thereof are merely provided for convenience of description and not intended to limit the present disclosure to the scope of the embodiments. It is understood that for those skilled in the art, after understanding the principle of the system, it may be possible to arbitrarily combine various modules to form a sub-system to connect with other modules without departing from the principle. In some embodiments, the obtaining module 210, the analysis module 220, and the determination module 230 may be different modules in one system or one module that implements the functions of two or more of modules. For example, each module may share one storage module, or each module may have its own storage module. Such deformations are within the protection scope of the present disclosure.

FIG. 3 is a flowchart illustrating an exemplary process for determining a target motion state according to some embodiments of the present disclosure. In some embodiments, the process for determining a target motion state may be performed by the processing device 120 or the system 200 for determining a target motion state. For example, the process 300 may be stored in a storage device (e.g., the storage device 150) in the form of a program or instruction, and the process 300 may be implemented when the processing device 120 or the system 200 for determining a target motion state executes the program or instruction. The schematic diagram illustrating operations of the process 300 presented below is illustrative. In some embodiments, the process may be accomplished using one or more additional operations not described and/or one or more operations not discussed. Additionally, the order of the operations of the process 300 illustrated in FIG. 3 and described below is not limiting.

In 301, first scanning data of a subject during a first scanning stage may be obtained. The first scanning data may correspond to a first FOV. In some embodiments, the operation 301 may be performed by the obtaining module 210 or the processing device 120.

In some embodiments, the subject may be affected by a physiological activity (e.g., breathing and heartbeat). For example, the motion state of the subject may change in response to breathing and/or heartbeat. For example, the breathing, heartbeat, etc., of the subject may cause organ displacement and/or organ expansion of tissues. When the subject is a lung, the lung may contract and relax when affected by the physiological activity. When the subject is a heart, the heart may be displaced when affected by the physiological activity. More descriptions regarding the subject may be found in FIG. 1 and the relevant descriptions thereof. In some embodiments, the subject may move regularly.

In some embodiments, the first scanning data may be acquired during a first time period in the first scanning stage. The subject may move regularly. The first time period may include one or more motion cycles of the subject. For example, if the subject is the heart, the motion cycle may be a cardiac cycle. As another example, when the subject is the lung, the motion cycle may be a period of time in which the lung undergoes contraction and relaxation, i.e., a breathing cycle. As yet another example, when the subject is a stomach and intestines, the motion cycle may be a gastrointestinal peristalsis cycle, etc.

The motion cycle refers to a period of time elapsed from a certain moment to a time when the displacement, velocity, acceleration, etc., of the subject fully or roughly recovers to the same or similar as at the certain moment.

In some embodiments, the first FOV may be the size of an FOV of a magnetic resonance (MR) scan performed on the subject. The first FOV may be an actual reconstructed FOV of the subject represented in the first scanning data. In some embodiments, the first FOV may cover a range of the subject where is excited by an MR pulse sequence (e.g., a phase encoding gradient) when the MR scan is performed on the subject. In some embodiments, the first FOV may cover a range in which the subject is located. For example, the first FOV may extend beyond four sides of the subject or a certain distance around the subject. As another example, the first FOV may be set at an edge of the range in which the subject is located.

In some embodiments, the first FOV may be set according to actual needs. For example, the first FOV may be set to a maximum range that can determine a condition of a certain subject according to diagnostic needs of a doctor.

In some embodiments, the first FOV may be limited by the hardware limitations of the magnetic resonance device.

In some embodiments, the first scanning data may include first image data that is acquired after the MR scan is performed on the subject. For example, the first scanning data may a fourth-dimensional (4D) image of the subject during the first scanning stage. As another example, the first scanning data may include a sequence of three-dimensional (3D) images including temporal dimension information. Each of the 3D images in the sequence may include a timestamp indicating the time when the 3D image is acquired by the MRI device. As another example, the first scanning data may include multiple sequences of two-dimensional (2D) images and each sequence of 2D images may form a 3D image of the subject at a certain time.

In some embodiments, the obtaining module 210 may obtain first raw data (i.e., first K-space data) of the subject during the first scanning stage acquired by the MRI device performing the MR scan (e.g., a 4D MR scan) on the subject through the MRI device (e.g., the MRI device 110), and obtain the first scanning data by performing image reconstruction based on the first raw data. Exemplary image reconstruction techniques may include, but are not limited to, a partial Fourier reconstruction technique, a parallel reconstruction technique, a compressed sensing reconstruction technique, a deep-learning-based reconstruction technique, etc.

In some embodiments, the first scanning data may include multiple sets of first images (also referred to as image sets) of the subject. Each set of the multiple sets of first images of the subject may include one or more first images that are reconstructed based on raw data acquired when the subject is in a same or similar motion state of one or more motion cycles. In other words, the one or more first images in the same set may represent the same or similar motion state of the subject. For example, the first time period may include a first motion cycle and a second motion cycle of the subject. A set of first images in the first scanning data may include image 1 and image 2. The image 1 may be reconstructed based on raw data of the subject acquired during a first sub-period in the first motion cycle and the image 2 may be reconstructed based on raw data of the subject acquired during a second sub-period in the second motion cycle. The first sub-period and the second sub-period may belong to the same or similar phase in the first motion cycle and the second motion cycle, such that the image 1 and the image 2 may represent the same or similar motion state of the subject. In some embodiments, the one or more first images in each set may be fused to determine a target first image representing a motion state of the subject at a time in a motion cycle.

Each set of the multiple sets of first images of the subject may correspond to a motion state of the subject in a motion cycle. As used herein, a set of first images corresponding to a motion state refers to the set of first image representing the motion state of the subject. In other words, the motion state of the subject may be represented in the set of first images via pixel values. Different sets of first images may correspond to different motion state. In one set of the multiple sets of first images of the subject corresponding to one motion state, a position and/or morphology of the subject may change slightly. In two sets of the multiple sets of first images of the subject corresponding to two different motion states, the position and/or morphology of the subject may change greatly or obviously.

The motion state corresponding to a set of first images may be represented by the position (i.e., a position of the subject at a certain moment of the motion cycle relative to the position of the subject at an initial moment of the motion cycle), the morphology (e.g., volume), etc., in the set of first images. In some embodiments, positions and/or morphological sizes in different ranges may be considered to be the different motion states.

For example, if the subject is the heart, the motion states during the cardiac cycle may be divided based on a change in the size of a volume of the heart and/or a change in a certain position at an edge of the heart. Merely by way of example, when the heart is in motion, the motion states during the heartbeat cycle may be divided into a mild beating state, a moderate beating state, a vigorous beating state, etc. In the mild beating state, the change in the size of the volume of the heart or the change in a certain position at the edge of the heart may be smaller than a first threshold corresponding to the mild beating state. In the moderate beating state, the change in the size of the volume of the heart or the change in a certain place at the edge of the heart is smaller than a second threshold corresponding to the moderate beating state and is greater than or equal to the first threshold. In the vigorous beating state, the change in the size of the volume of the heart or the change in a certain position at the edge of the heart may be smaller than a third threshold corresponding to the vigorous beating state and is greater than or equal to the second threshold. The first threshold, the second threshold, and the third threshold may be system setting values, system default values, artificially set values, etc.

As another example, if the subject is the lung, the motion states of the lung during a motion cycle of contraction and expansion may be divided based on a change in a size of a volume of the lung or a change in a certain position at an edge of the lung. Merely by way of example, the motion states of the lung during the motion cycle of contraction and expansion may be divided into a primary contraction state, a secondary contraction state, etc., and/or a primary expansion state, a secondary expansion state, etc. For the manner in which the motion states are divided, please refer to the manner in which the motion states during the heartbeat cycle are divided above, which is not repeated herein.

In some embodiments, the motion state may be represented by a velocity, an acceleration, a motion direction, a motion amplitude, etc., of the subject. In some embodiments, the velocity, the acceleration, the motion direction, the motion amplitude being in different ranges may be considered as different motion states.

The multiple sets of first images may be generated by dividing the first image data, e.g., the 4D image, the sequence of 3D images, the multiple sequences of 2D images. For example, each set of the multiple sets of first images of the subject may include one or more 3D images of the subject by dividing the 4D image or the sequence of 3D images. As another example, each set of the multiple sets of images of the subject may include one or more sequences of 2D images of the subject by dividing the multiple sequences of 2D images.

As shown in FIG. 4, in some embodiments, the obtaining module 210 may obtain the first image data of the subject during the first scanning stage and obtain the multiple sets of first images of the subject by performing three-dimensional (3D) state segmentation based on the first image data.

In some embodiments, the obtaining module 210 may segment the first image data into the multiple sets of first images of the subject by performing three-dimensional state segmentation on the first image data. In some embodiments, the obtaining module 210 may perform the three-dimensional state segmentation on the first image data through image matching. For example, the obtaining module 210 may obtain one or more reference images corresponding to the different motion states, and match the first images in the first image data with the reference images corresponding to the different motion states, thereby segmenting the first image data into the multiple sets of first images of the subject corresponding to the different motion states. The first images in the first image data that are matched with a reference image corresponding to a motion state may be designated as a set of first images corresponding to the motion state.

In some embodiments, the obtaining module 210 may segment the first image data into the multiple sets of first images through image comparison. For example, the obtaining module 210 may slidingly intercept a plurality of time segments, perform similarity comparison on the plurality of first images corresponding to each time segment, designate first images whose image similarity is smaller than a similarity threshold to form a set of first images corresponding to a motion state, thereby segmenting the first image data into the multiple sets of 3D images. The similarity threshold may be a system default value, an empirical value, an artificially preset value, or the like, or any combination thereof, which may be set according to actual needs.

In some embodiments, the processing device may segment the first k-space data into multiple portions and each of the multiple portions of the first k-space data may correspond to a motion state. For example, the first physiological signal of the subject and the first k-space data may be acquired simultaneously and the first k-space data may be divided into the multiple portions based on the first physiological signal. As a further example, the processing device may divide the first physiological signal into physiological phases (i.e., motion states) based on the characteristics of the first physiological signal. Each of the first physiological phases may correspond to a time phase and the processing device may divide the first k-pace data into the multiple portions according to the time phase corresponding to each of the first physiological phases. Then the processing device may reconstruct the multiple sets of first images based on the multiple portions of the first k-space data. For example, each of the multiple portions of the first k-space data may be used to reconstruct one or more first images corresponding to a motion state.

In some embodiments, the processing device may divide the first image data according to a default time interval.

In some embodiments, the processing device may fuse the first images in each set of the multiple sets of first images to obtain a target first image corresponding to a motion state, and designate the target first image as the first image in the set of first images.

In some embodiments, the processing device may identify the subject represent in each of the multiple sets of first images and/or mark the identified subject. The processing device may determine the motion state of the subject corresponding to the set of first images based on the identified subject.

The embodiments of the present disclosure do not have any special limitation on the way of three-dimensional state segmentation, and it is sufficient to adopt the operation known to those skilled in the art.

In 302, second scanning data of the subject during the first scanning stage may be obtained. The second scanning data may correspond to a second FOV. In some embodiments, the operation 302 may be performed by the obtaining module 210. In some embodiments, the first scanning data may be acquired during the first time period in the first scanning stage and the second scanning data may be acquired during a second time period during the first scanning stage. In some embodiments, the first time period and the second time period may be consecutive.

In some embodiments, the second scanning data may be acquired based on a quick MR scan technique.

In some embodiments, the obtaining module 210 may perform the quick magnetic resonance scan on the subject in various ways. For example, the quick magnetic resonance scan may be performed on the subject by reducing the amount of signal data that needs to be obtained, shortening the time for obtaining an echo signal, etc.

In some embodiments, the obtaining module 210 may increase the imaging speed of the magnetic resonance scan by reducing the count of phase encoding steps (i.e., reducing the obtaining of echo signals), increasing a pixel matrix in a phase encoding direction, and reducing a spatial resolution in the phase encoding direction.

In some embodiments, the obtaining module 210 may increase the imaging speed of the magnetic resonance scan by reducing the number of times of repeated obtaining performed at each phase encoding step.

In some embodiments, the obtaining module 210 may increase the imaging speed of the magnetic resonance scan using a partial K-space technique. For example, data that fills half of the K-space or more than half of the K-space may be obtained, and the rest of the K-space that is not filled may be filled using the principle that the K-space is symmetric in the phase encoding direction.

In some embodiments, the phase encoding direction may be adjusted when the quick magnetic resonance scan is performed. For example, the phase encoding direction may be set on an anatomical short axis of a human body.

The descriptions regarding the manner of quick magnetic resonance scan are merely provided for the purpose of illustration, and are not intended to limit the scope of the present disclosure.

In some embodiments, the second FOV may not cover the range of the subject where is excited by an MR pulse sequence (e.g., a phase encoding gradient) when the MR scan is performed on the subject to acquire the second scanning data. For example, the second FOV may be smaller than the range in which the subject is located. The second FOV may be smaller than the first FOV. The second FOV may be set to be smaller than the first FOV, so that the second scanning data may have a stronger signal strength of low-frequency components in the center region of the k-space and high-frequency components in the edge regions of the K-space compared to the first scanning data, which may provide a better representation of the motion state of the subject. In some embodiments, since the second FOV is smaller than the range in which the subject is located, image wrap may be generated in the second scanning data.

The second FOV may be determined in various ways. In some embodiments, the second FOV may be preset by a system (e.g., the system 200 for determining a target motion state) or artificially. For example, the size of the second FOV may be preset by the system or artificially according to prior knowledge or historical data.

In some embodiments, the second FOV may be determined based on the first FOV. For example, the obtaining module 210 may obtain the second FOV by performing an operation such as an equal-proportional reduction on the size of the first FOV.

In some embodiments, the second FOV may be determined based on the organ type of the subject. For example, the system may determine the second FOV to be applied to the subject according to an actual organ type of the subject based on a correspondence between different organ types and different FOVs.

In some embodiments, the second FOV may be determined based on the first scanning data and a preset condition.

The preset condition may be in various forms. In some embodiments, the preset condition may include setting a size of the second FOV to make the second FOV satisfy a motion type of the subject. The motion type of the subject refers to the motion direction of the subject, for example, moving up and down, moving left and right, etc. In some embodiments, the motion type of the subject may be determined based on the first scanning data. For example, the motion type (i.e., the motion direction) of the subject may be determined based on adjacent 3D images.

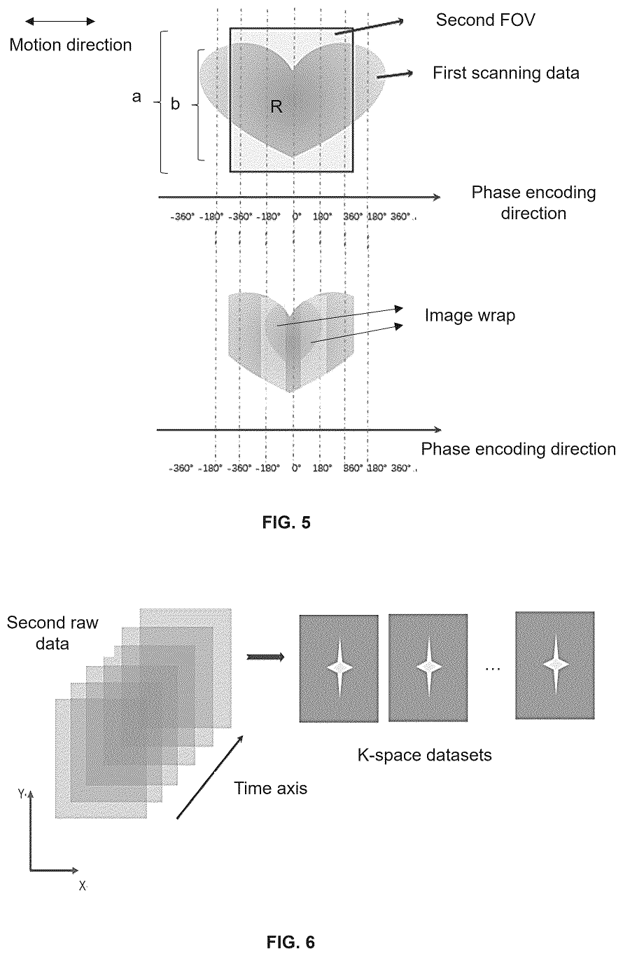

In some embodiments, satisfying the motion type of the subject means that the length of the side of the second FOV perpendicular to the motion direction of the subject is the same as or similar to the length of the side of the subject perpendicular to the motion direction of the subject. As shown in FIG. 5, R denotes the subject, “a” denotes a side of the second FOV that is perpendicular to the motion direction of the subject R, and “b” denotes a side of the subject R that is perpendicular to the motion direction of the subject R. Therefore, satisfying the motion type of the subject means that the length of the “a” side of the second FOV is the same as or similar to the length of the “b” side of the subject R.

In some embodiments, the preset condition may include setting the size of the first FOV and/or the second FOV such that an image obtained based on the first scanning data does not include image wrap, and an image obtained based on the second scanning data includes image wrap. As shown in FIG. 5, the second scanning data corresponding to the second FOV may include the image wrap.

In some embodiments, the obtaining module 210 may adjust the size of the first FOV to make that the image obtained based on the first scanning data does not include the image wrap, and the image obtained based on the second scanning data includes the image wrap, and use the adjusted size of the first FOV as the size of the second FOV.

In some embodiments, the preset condition may include: setting the size of the second FOV such that the temporal resolution of the second scanning data during the first scanning stage satisfies a preset temporal resolution condition and/or the image wrap appears in a direction in which motion of the subject has the greatest impact on the imaging.

In some embodiments, the preset temporal resolution condition may be that the temporal resolution of the second scanning data during the first scanning stage is greater than or equal to the temporal resolution threshold. The temporal resolution threshold may be a system default, an empirical value, an artificial preset value, or the like, or any combination thereof, which may be set according to actual needs, and may not be limited in the present disclosure.

In some embodiments, the direction in which the motion of the subject has the greatest impact on the imaging may be the same as the motion direction of the subject. In some embodiments, the direction in which the motion of the subject has the greatest impact on the imaging may be the phase encoding direction. In some embodiments, the direction in which the motion of the subject has the greatest impact on the imaging may be a frequency encoding direction.

In some embodiments, the system may adjust the size of the first FOV to make that the temporal resolution of the second scanning data during the first scanning stage satisfies the preset temporal resolution condition and/or the image wrap appears in the direction in which motion of the subject has the greatest impact on the imaging, and use the adjusted size of the first FOV as the second FOV.

In some embodiments, alternatively or additionally, the preset condition may include setting the size of the second FOV to make that the spatial resolution of the second scanning data satisfies a preset spatial resolution condition. In some embodiments, the preset spatial resolution condition may be that a spatial resolution of the second scanning data during the first scanning stage is smaller than a spatial resolution threshold. The spatial resolution threshold may be a system default, an empirical value, an artificially preset value, or the like, or any combination thereof, which may be set according to actual needs, and may not be limited in the present disclosure.

The preset condition may also be in other feasible forms, which may be set according to actual needs.

In some embodiments, the obtaining module 210 may adjust the size of the first FOV to make that the spatial resolution of the second scanning data satisfies the preset spatial resolution condition, and use the adjusted size of the first FOV as the second FOV.

In some embodiments, the second FOV may be related to at least one of a scanning parameter, a subject feature, or a resolution requirement associated with the second FOV.

The scanning parameter associated with the second FOV refers to a scanning parameter used by the MRI device to acquire the second scanning data. In some embodiments, the scanning parameter associated with the second FOV may include an MR pulse sequence, a phase encoding direction, a frequency encoding direction, a layer thickness setting, etc. More descriptions regarding the MR pulse sequence may be found in the relevant descriptions of FIG. 1.

The scanning parameter associated with the second FOV may be determined in various ways. In some embodiments, the scanning parameter associated with the second FOV may be preset by a system (e.g., the system 200 for determining a target motion state) or artificially. For example, the scanning parameter may be preset by the system or artificially according to prior knowledge or historical data.

The subject feature refers to a feature related to the subject. In some embodiments, the subject feature may include an organ type of the subject, a size of the subject, the position of the subject, or the like, or a combination thereof. The size of the subject may include the length of the subject in a direction perpendicular to the motion direction of the subject, and the length of the subject in a direction parallel to the motion direction of the subject.

The subject feature may be determined in various ways. In some embodiments, the subject feature may be determined through detection of the system (e.g., the system 200 for determining a target motion state) or through human input.

The resolution requirement refers to a requirement related to a resolution when the second scanning data is acquired by the imaging device. The resolution may include the temporal resolution and/or the spatial resolution. Accordingly, the resolution requirement may include a temporal resolution requirement and/or a spatial resolution requirement. In some embodiments, the temporal resolution requirement may include that the temporal resolution is smaller than a first preset threshold. In some embodiments, the spatial resolution requirement may include that the spatial resolution is smaller than a second preset threshold. The first preset threshold and/or the second preset threshold may be a system default value, an empirical value, an artificially preset value, or the like, or any combination thereof, which may be set according to actual needs, and may not be limited by the present disclosure.

In some embodiments, the resolution requirement may be determined through human input.

In some embodiments, the obtaining module 210 may determine the second FOV in various ways based on at least one of the scanning parameter, the subject feature, or the resolution requirement associated with the second FOV. In some embodiments, the obtaining module 210 may determine the second FOV by querying a preset table based on at least one of the scanning parameter, the subject feature, or the resolution requirement associated with the second FOV.

In some embodiments, the preset table may include a correspondence between a plurality of parameter combinations and a plurality of reference FOVs. A parameter combination refers to a combination including at least one of the scanning parameter, the subject feature, or the resolution requirement associated with a reference FOV.

In some embodiments, the preset table may be preset by the system (e.g., the system 200 for determining a target motion state). In some embodiments, the system may preset a plurality of parameter combinations. For each parameter combination, the system may determine, through testing, the reference FOV corresponding to the parameter combination. For example, for each parameter combination, the system may obtain different test scanning data by performing a test scan using different FOVs, and take an FOV corresponding to test scanning data that satisfies a preset condition as the reference FOV corresponding to the parameter combination. The scan performed during the testing may be the quick magnetic resonance scan, and the test scanning data obtained may be scanning data obtained after the quick magnetic resonance scan is performed.

More descriptions regarding the preset condition may be found above.

In some embodiments, the obtaining module 210 may determine the second FOV through a prediction model based on the scanning parameter, the subject feature, and/or the resolution requirement associated with the second FOV.

The prediction model refers to a model configured to determine the second FOV. In some embodiments, the prediction model may be a trained machine learning model. For example, the prediction model may include a convolutional neural networks (CNN) model, a neural networks (NN) model, or other customized model structures, or the like, or any combination thereof.

In some embodiments, an input of the prediction model may include the scanning parameter, the subject feature, and/or the resolution requirement associated with the second FOV, and an output of the prediction model may be the second FOV.

In some embodiments, the prediction model may include at least 800 or more multiplication operations in a single execution. In some embodiments, the prediction model may be at least partially executed by a graphics processing unit (GPU).

In some embodiments, the prediction model may be obtained through training. In some embodiments, the prediction model may be obtained through training of the system (e.g., the system 200 for determining a target motion state) and stored in a storage device (e.g., a computer storage medium).

In some embodiments, the training of the prediction model may include obtaining a training dataset including a plurality of first training samples and a first label corresponding to each first training sample, and performing a plurality of iterations until a termination condition is satisfied. Each of the plurality of iterations may include inputting a first training sample into an initial prediction model, and obtaining a model prediction output corresponding to the first training sample, determining a value of a loss function by substituting the model prediction output corresponding to the first training sample and the first label of the first sample into a formula of a predefined loss function; and inversely updating a model parameter of the initial prediction model based on the value of the loss function. The operation may be performed in various ways. For example, the updating may be performed based on a gradient descent algorithm. When a termination condition is satisfied, the iterations may be completed, and a trained prediction model may be obtained. The termination condition may include the loss function converging, a count of rounds of iteration reaching a threshold, etc.

In some embodiments, a first training sample may include a sample scanning parameter, a sample subject feature, and/or a sample resolution requirement. The first label corresponding to the first training sample may be a reference FOV. In some embodiments, the first training sample may be obtained based on historical data. The first label corresponding to the first training sample may be obtained by manual or systematic labeling. In some embodiments, historical scanning parameters, historical subject features, and historical resolution requirements of a large amount of historical scanning data may be used as the first training sample, and a historical second FOV may be labeled as the first label.

In some embodiments, an input of the prediction model may also include the first FOV. That is, at this point, the input of the prediction model may include the scanning parameter, the subject feature, the resolution requirement associated with the second FOV, and the first FOV, and the output of the prediction model may be the second FOV.

When the first FOV is used as the input of the prediction model, a historical first FOV may be added to the first training sample during model training to train the prediction model.

In some embodiments, the input of the prediction model may also include the first scanning data. That is, at this point, the input of the prediction model may include the scanning parameter, the subject feature, the resolution requirement associated with the second FOV, and the first scanning data, and the output of the prediction model may be the second FOV.

In some embodiments, the prediction model may include a plurality of processing layers. For example, the prediction model may include a first feature extraction layer and a first prediction layer. In some embodiments, the first feature extraction layer may include a model such as a CNN model, and the first prediction layer may include a model such as an NN model.

In some embodiments, the first feature extraction layer may determine a motion feature of the subject based on the first scanning data. The motion feature refers to feature data used to describe the motion state of the subject. For example, the motion feature may include a motion direction, an overall magnitude of motion, etc. More descriptions regarding the motion state may be found in the operation 301 and the relevant descriptions thereof.

In some embodiments, the first prediction layer may determine the second FOV based on the scanning parameter, the subject feature, the resolution requirement associated with the second FOV, and the motion feature.

In some embodiments, the first feature extraction layer and the first prediction layer may be obtained by training separately.

In some embodiments, the system may obtain a trained first feature extraction layer by updating a model parameter in various ways based on a plurality of second training samples with second labels. For example, the first feature extraction layer may be trained based on the gradient descent algorithm. Merely by way of example, the plurality of second training samples with second labels may be input into an initial first feature extraction layer, a loss function may be constructed based on the second labels and a result of the initial first feature extraction layer, and a parameter of the initial first feature extraction layer may be iteratively updated based on the loss function. The training may be completed when the loss function of the initial first feature extraction layer satisfies a termination condition, and a trained first feature extraction layer may be obtained.

In some embodiments, the second training sample may include sample first scanning data obtained by performing a 4D MR scan on the sample subject, and the second label corresponding to the second training sample may be a reference motion feature of the sample subject. In some embodiments, the second training sample may be obtained based on historical data. The second label corresponding to the second training sample may be obtained by manual or systematic labeling. For example, the motion feature of the sample subject may be obtained by performing data processing and analysis (e.g., image registration, optical flow approach, etc.) on the sample first scanning data or the sample first raw data.

In some embodiments, the system may obtain a trained first prediction layer by updating a model parameter in various ways based on a plurality of third training samples with third labels. The training process of the first prediction layer is similar to the training process of the first feature extraction layer. More descriptions may be found above. In some embodiments, the third training sample may include a sample scanning parameter, a sample subject feature, a sample resolution requirement, and a sample motion feature of the sample subject when the quick magnetic resonance scan is performed on the sample subject, and the third labeling may be the second FOV. In some embodiments, the third training sample may be obtained based on historical data. The third label corresponding to the third training sample may be obtained by manual or systematic labeling. The third label is labeled in a manner similar to the manner in which the first label is labeled. More descriptions may be found above.

In some embodiments, the input of the prediction model may also include a test FOV and test scanning data. That is, at this point, the input of the prediction model may include the scanning parameter, the subject feature, and the resolution requirement associated with the second FOV, the test FOV, and the test scanning data, and the output of the prediction model may be the second FOV.

The test FOV refers to an FOV when the test scan is performed on the subject. In some embodiments, the test scan may be a process for performing a quick magnetic resonance scan on the subject using a test FOV. The scanning data obtained by the test scan may be the test scanning data.

The test FOV may be determined in various ways. In some embodiments, the system (e.g., the system 200 for determining a target motion state) may use the first FOV as the test FOV. In some embodiments, the system may use an FOV during a process of adjusting the first FOV based on a preset requirement as the test FOV. In some embodiments, the system may use the second FOV determined by the preset table as the test FOV. The embodiments of the present disclosure do not have any special limitation on the way of obtaining the test FOV, and it is sufficient to adopt the operation known to those skilled in the art.

In some embodiments, after determining the test FOV, the system may obtain the test scanning data by performing the quick magnetic resonance scan on the subject using the test FOV.

In some embodiments, when the input of the prediction model includes the scanning parameter, the subject feature, and the resolution requirement associated with the second FOV, the test FOV, and the test scanning data, the prediction model may include a plurality of processing layers. For example, the prediction model may include a second feature extraction layer and a second prediction layer. In some embodiments, the second feature extraction layer may include a model such as a CNN model, and the second prediction layer may include a model such as an NN model.

In some embodiments, the second feature extraction layer may determine a test scanning feature based on the test scanning data. The test scanning feature refers to feature data used to describe test image data. The test image data refers to image data obtained by reconstruction based on the test scanning data. For example, the test scanning feature may include the size, intensity, etc., of the image wrap of the test image data.

In some embodiments, the second prediction layer may determine the second FOV based on the scanning parameter, the subject feature, and the resolution requirement associated with the second FOV, the motion feature, the test FOV, and the test scanning feature.

In some embodiments, the second feature extraction layer and the second prediction layer may be obtained by training separately.

In some embodiments, the system may obtain a trained second feature extraction layer by updating a model parameter based on a plurality of fourth training samples with fourth labels in various ways. The training process of the second feature extraction layer is similar to the training process of the first feature extraction layer. More descriptions may be found above.

In some embodiments, a fourth training sample may include sample test scanning data when the test scan is performed on the sample subject, and the fourth label corresponding to the fourth training sample may be the test scanning feature of the sample subject. In some embodiments, the fourth training sample may be obtained based on historical data. The fourth label corresponding to the fourth training sample may be obtained by manual or systematic labeling. For example, the fourth label may be obtained by systematic or human labeling based on a reconstruction result of the sample test scanning data.

In some embodiments, the system may obtain a trained second prediction layer by updating a model parameter in various ways based on a plurality of fifth training samples with fifth labels. The training process of the second prediction layer is similar to the training process of the first feature extraction layer. More descriptions may be found above. In some embodiments, a fifth training sample may include a sample scanning parameter, a sample subject feature, a sample resolution requirement, and a sample test FOV, and a sample test scanning feature when the quick magnetic resonance scan is performed on the sample subject, and the fifth label may be a reference FOV. In some embodiments, the fifth training sample may be obtained based on historical data. The fifth label corresponding to the fifth training sample may be obtained by manual or systematic labeling. The fifth label is labeled in a manner similar to a manner in which the first label is labeled. More descriptions may be found above.

Several adjustments may be required to actually determine the second FOV until a final second FOV is obtained, such that the second scanning data meets the preset requirement. In some embodiments of the present disclosure, the test FOV and the test scanning data may be input into the prediction model, and the system may execute the process of first obtaining the test scanning data once using the test FOV for a test and obtaining the second FOV by adjusting the test FOV according to the performance of the test scanning data.

In other embodiments, the input of the prediction model may further include a plurality of test FOVs and a plurality of pieces of test scanning data. The plurality of test FOVs may be in a one-to-one correspondence with the plurality of pieces of test scanning data. That is, at this point, the input of the prediction model may include the scanning parameter, the subject feature, the resolution requirement associated with the second FOV, the plurality of test FOVs, and the plurality of pieces of test scanning data when the second scanning data is acquired by the imaging device, and the output of the prediction model may be the second FOV.

In some embodiments of the present disclosure, the plurality of test FOVs and the plurality of pieces of test scanning data may be input into the prediction model, the prediction model may determine whether the test scanning data corresponding to each test FOV meets the preset requirement or not, so that a most suitable test FOV corresponding to the test scanning data may be determined as the second FOV.

In some embodiments, the second scanning data may include k-space data including temporal dimension information.

In some embodiments, the second scanning data may include one or more K-space datasets of the subject acquired during one or more motion cycles in the first scanning stage. The subject may include one or more motion states during each motion cycle, and each K-space dataset may correspond to one motion state of the one or more motion states. In one K-space dataset corresponding to a motion state, the position or morphology of the subject may change slightly. In two K-space datasets corresponding to different motion states, the position and/or morphology of the subject may change greatly or obviously.

In some embodiments, the length of the acquisition duration corresponding to each K-space dataset in the second scanning data may be the same. In some embodiments, the length of the acquisition duration corresponding to each K-space dataset in the second scanning data may be different. In some embodiments, the length of the acquisition duration corresponding to each set of first images in the first scanning data may be the same. In some embodiments, the length of the acquisition duration corresponding to each set of first images in the first scanning data may be different. In some embodiments, the length of the acquisition duration corresponding to each set of first images in the first scanning data may be the same as the length of the acquisition duration corresponding to each K-space dataset in the second scanning data. In some embodiments, the length of the acquisition duration corresponding to each set of first images in the first scanning data may be different from the length of the acquisition duration corresponding to each K-space dataset in the second scanning data.

In some embodiments, a count of K-space datasets in the second scanning data may be the same as a count of sets of 3D images in the first scanning data. As shown in FIG. 6, in some embodiments, the obtaining module 210 may obtain second raw data (i.e., second k-space data) of the subject, and obtain the at least one K-space dataset by dividing the second raw data.

In some embodiments, the second raw data may be acquired after the quick MR scan is performed on the subject.

In some embodiments, the second raw data may correspond to the second FOV. Accordingly, the obtaining module 210 may obtain the raw data (e.g., the second K-space data) of the subject during the second time period by performing the quick MR scan on the subject in the second FOV. The obtained raw data may be the second raw data.

In some embodiments, each piece of k-space data in the second scanning data may be designate as a k-space dataset. As used herein, each piece of k-space data may refer to k-space data acquired during a duration that an MR pulse sequence is performed for once.

In some embodiments, the processing device may determine a similarity between two pieces of k-space data and designate two pieces of k-space data whose similarity degrees exceeds a threshold into one same k-space dataset.

In some embodiments, the obtaining module 210 may divide the second raw data into the one or more K-space datasets by dividing the second raw data based on K-space data variation feature.

In some embodiments, the K-space data variation feature may include differences in high-frequency and low-frequency signal distribution.

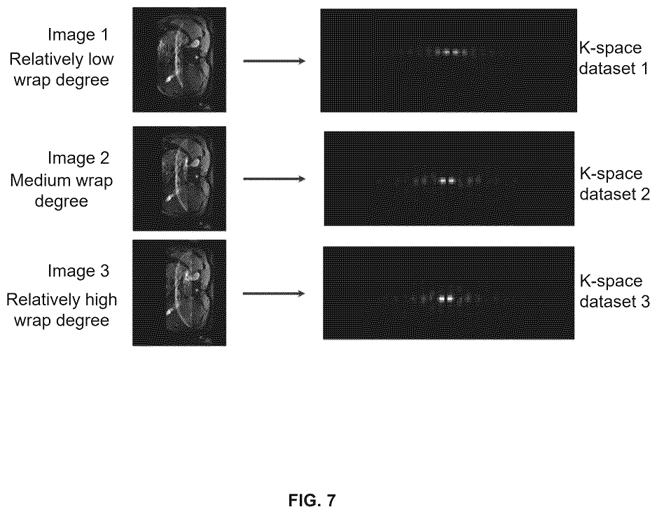

The differences in high-frequency and low-frequency signal distribution may involve in the K-space data acquired under the FOV being less than the size of the subject to be excited or scanned. The size and/or the position of the subject to be excited by the phase encoding gradient may be change during the motion of the subject. The change of the size and/or the position of the subject may cause the covered region of the FOV on the subject change. The differences in high-frequency and low-frequency signal distribution in the K-space data may cause image wrap in a reconstructed image. As shown in FIG. 7, left images 1, 2, 3 shows the image wrap with different wrap degrees. Image 1 shows a relatively low wrap, Image 2 shows a medium wrap degree, and Image 3 shows a relatively high wrap degree. Images 1, 2, 3 were respectively reconstructed based on k-space dataset 1, k-space dataset 2, and k-space dataset 3, and the differences between the K-space datasets 1, 2, 3 and a reference K-space dataset is shown on the right of FIG. 7. The reference K-space dataset refers to a K-space dataset whose reconstructed MR image has no image wrap. As shown in FIG. 7, for Image 3 having a relatively strong wrap degree of image wrap, low-frequency components in the center region of the k-space dataset 3 and high-frequency components in the edge regions of the k-space dataset 3 have more enhancements relative to the reference K-space dataset For Image 1 having a relatively small wrap degree of image wrap, the low-frequency components in the center region of the k-space dataset 1 and high-frequency components in the edge regions of the k-space dataset 1 have few enhancements relative to the reference K-space dataset. That is to say, the stronger the signal strength of the low-frequency components in the center region and high-frequency components in the edge regions of a K-space dataset, the stronger the wrap degree in an image reconstructed using the K-space dataset.

Accordingly, the obtaining module 210 may divide the second raw data into one or more K-space datasets by distinguishing the differences in high-frequency and low-frequency components in the second raw data. In one K-space dataset corresponding to one motion state, the signal strength of the low-frequency components in the center region and high-frequency components in the edge regions of the K-space data obtained at different moments may change slightly or not obviously. In two K-space datasets corresponding to different motion states, the signal strength of the low-frequency components in the center region and high-frequency components in the edge regions of the different K-space datasets may change greatly or significantly.

For example, K-space data of an enhancement (which may be an average enhancement of all low-frequency signals) of a low-frequency signal strength in the edge regions within one same range in the plurality of pieces of K-space data in the second raw data may be divided into one same K-space dataset. As another example, K-space data of an enhancement (which may be an average enhancement of all high-frequency signals) of a high-frequency signal strength in the center region within one same range in the plurality of pieces of K-space data in the second raw data may be divided into one same K-space dataset. As yet another example, the K-space data of the enhancement of the low-frequency signal strength in the edge regions within one same range and the K-space data of the high-frequency signal strength in the center region within one same range in the plurality of pieces of K-space data in the second raw data may be divided into one same K-space dataset.

In some embodiments, the processing device may segment the second k-space data into multiple portions and each of the multiple portions of the second k-space data may correspond to a motion state. For example, the second physiological signal of the subject and the second k-space data may be acquired simultaneously and the second k-space data may be divided into the multiple portions based on the second physiological signal. As a further example, the processing device may divide the second physiological signal into second physiological phases (i.e., motion states) based on characteristics of the second physiological signal. Each of the second physiological phases may correspond to a time phase and the processing device may divide the second k-pace data into the multiple portions according to the time phase corresponding to each of the second physiological phases. Each of the multiple portions of the second k-space data may be designated as a k-space dataset corresponding to a motion state. The second physiological phases may be determined based on the first physiological phases, such that a correspondence between the first scanning data and the second scanning data may be determined.

In some embodiments, the processing device may fuse the k-space data in each k-space dataset to obtain a target k-space data corresponding to a motion state.

In 303, a target correspondence between the first scanning data and the second scanning data of the subject corresponding to a same motion state during the first scanning stage may be determined based on the first scanning data and the second scanning data. In some embodiments, the operation 303 may be performed by the analysis module 220. As used herein, determining the target correspondence between the first scanning data and the second scanning data refers to determining a portion of the first scanning data and a portion of the second scanning data that correspond to the same motion state.

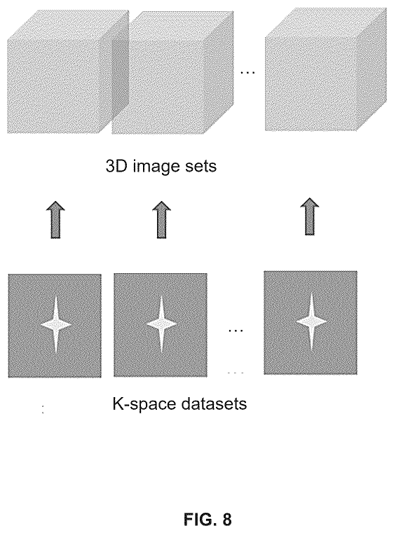

The target correspondence may reflect a correspondence between one or more pieces of data of the first scanning data obtained in the first FOV and one or more pieces of data of the second scanning data obtained in the second FOV. For example, the first scanning data may include multiple sets of first images and the second scanning data includes multiple K-space datasets. As shown in FIG. 8, the target correspondence may include the target correspondence between the multiple sets of first images and the K-space datasets. In other words, the target correspondence may show each set of first images and a corresponding k-space dataset that correspond to the same motion state.

The target correspondence may be obtained in various ways. In some embodiments, the analysis module 220 may determine a motion state corresponding to each set of first images and a motion state corresponding to each K-space dataset, and determine the target correspondence between the each set of first images and the each K-space dataset based on the motion states For example, if each of the at least one set of first images may correspond to one motion state of the subject during the first scanning stage, and each K-space dataset may correspond to one motion state of the subject during the first scanning stage, the analysis module 220 may determine the target correspondence by associating a set of first images with a K-space dataset with a same motion state. As another example, the analysis module 220 may label, based on the motion states, the multiple sets of first images and the K-space datasets, respectively, and associate a set of first images with a K-space dataset with a same label by analyzing labels of different sets of 3D images and K-space datasets to determine the target correspondence. Merely by way of example, when the subject is a lung, a label type may include “primary inspiratory state (e.g., less inspiratory),” “secondary inspiratory state (e.g., more inspiratory),” “tertiary inspiratory state (e.g., (e.g., inspiratory limi),” etc.

In some embodiments, the processing device may reconstruct a second image (e.g., second image) based on each of the k-space datasets. The processing device may determine a similarity between each of the second images and each of the 3D images (i.e., first images) in the first scanning data and determine the target correspondence between the first scanning data and the second scanning data based on the similarities between the second images and the first images. For example, the processing device may designate a first image and a second image who has a maximum similarity with the first image as a corresponding first image and second image that corresponding to the same motion state.

In some embodiments, the analysis module 220 may determine, based on the at least one set of 3D images and the at least one K-space dataset, a target correspondence between the K-space datasets of the subject in the at least one motion state during the first scanning stage and the set of 3D images through a matching model.

The matching model may be a machine learning model. In some embodiments, the matching model may include various feasible models such as a recurrent neural network (RNN) model, a deep neural network (DNN) model, a convolutional neural network (CNN) model, or the like, or any combination thereof.

In some embodiments, an input of the matching model may include the sets of 3D images and the K-space datasets, and an output of the matching model may include the target correspondence of the subject in at least one motion state during the first scanning stage.

In some embodiments, the matching model may be obtained by training a large number of training samples with labels. In some embodiments, the training sample may include at least one sample three-dimensional scanning image and at least one sample K-space dataset, and the label may include an actual target correspondence corresponding to the training sample. In some embodiments, the training sample may be obtained based on historical data, and the label may be determined by systematic or human labeling. For example, the sample 3D images and the sample K-space dataset for which there is a correspondence may be determined by the system or artificially through image matching or image comparison (descriptions may be found above), and the actual target correspondence between the at least one sample three-dimensional scanning image and the at least one sample K-space dataset may be obtained.

In some embodiments of the present disclosure, the target correspondence may be determined using the machine learning model, the pattern may be found from a large amount of data using the self-learning capability of machine learning, and the correspondence between the three-dimensional scanning image and the K-space dataset may be obtained, thereby improving the accuracy and efficiency of determining the target correspondence.

In some embodiments, the analysis module 220 may determine a first correspondence between the first scanning data and at least one motion state during the first scanning stage; and determine, based on the second scanning data and the first correspondence between the first scanning data and at least one motion state during the first scanning stage, the target correspondence.

The first correspondence refers to a correspondence between a set of first images and a motion state. The subject may include at least one motion state during the motion cycle, and each set of first images may correspond to one motion state of the at least one motion state. The motion state may be denoted by the position of the subject presented in each of the 3D images. The motion state corresponding to each set of the one or more sets of first images may be determined by identifying the subject represented in each of the one or more sets of first images and determining the position of the subject based on the identified subject in each of the one or more sets of first images.

In some embodiments, the analysis module 220 may perform image reconstruction based on the second scanning data; perform image matching based on a plurality of second images with the multiple sets of first images in the first scanning data; and obtain an image correspondence between the plurality of second images and the multiple sets of first images. The analysis module 220 may determine, based on the image correspondence, and a correspondence between the multiple K-space datasets in the second scanning data and the plurality of second images, a correspondence (i.e., the target correspondence) between the multiple K-space datasets and the multiple sets of first images.

In some embodiments, the analysis module 220 may determine, based on the second scanning data, a second correspondence between the second scanning data and at least one motion state during the first scanning stage; and determine, based on the first correspondence and the second correspondence, the target correspondence.

The second correspondence refers to a correspondence between a K-space dataset and a motion state. The subject may include at least one motion state during the motion cycle, and each K-space dataset may correspond to one motion state of the at least one motion state. The motion state corresponding to each k-space dataset may be determined by reconstructing a second image based on the k-space dataset, identifying the subject represented in the second image, and determining the position of the subject based on the identified subject in the second image.

In some embodiments, the analysis module 220 may determine, through a motion state prediction model, the motion state corresponding to each K-space dataset in the second scanning data, and determine a correspondence between the each K-space dataset and the motion state as the second correspondence.

The motion state prediction model may be a machine learning model. In some embodiments, the motion state prediction model may include various feasible models such as an RNN model, a DNN model, a CNN model, or the like, or any combination thereof.

In some embodiments, an input of the motion state prediction model may include at least one K-space dataset or at least one K-space dataset, and an output of the motion state prediction model may be a motion state corresponding to the at least one K-space dataset or a motion state corresponding to the at least one K-space dataset.

In some embodiments, the motion state prediction model may be obtained by training a large number of training samples with labels. In some embodiments, the training sample may include at least one K-space dataset or at least one set of sample K-space datasets, and the label may include a motion state corresponding to the at least one K-space dataset or a motion state corresponding to the at least one set of sample K-space datasets. In some embodiments, the training sample may be obtained based on historical data, and the label may be determined by systematic or human labeling. For example, the motion state corresponding to the at least one K-space dataset or the motion state corresponding to the at least one set of sample K-space datasets may be determined by the system or artificially through the image registration (descriptions may be found above).

In some embodiments of the present disclosure, the motion state corresponding to the K-space dataset may be determined using the machine learning model, the pattern may be found from a large amount of data using the self-learning capability of machine learning, and the corresponding between the motion state and the K-space dataset may be obtained, thereby improving the accuracy and efficiency of determining the second correspondence.

In some embodiments, after determining the second correspondence, the analysis module 220 may determine, based on the first correspondence and the second correspondence, the target correspondence between the set of the first images in the first scanning data and the set of the K-space datasets in the second scanning data. For example, the analysis module 220 may obtain the target correspondence by designating the K-space dataset and set of first images corresponding to the same motion state be in a one-to-one correspondence based on the first correspondence and the second correspondence.

In 304, the target motion state of the subject during the second scanning stage may be determined based on the target correspondence between the first scanning data and the second scanning data. In some embodiments, the operation 304 may be performed by the determination module 230.

In some embodiments, the second scanning stage may be a real-time scanning period. The second scanning stage may be different from the first scanning stage.

In some embodiments, the second scanning stage may be a scanning period for determining, adjusting, and/or updating a treatment plan (e.g., a radiotherapy plan). In some embodiments, the second scanning state may be a scanning period before performing the treatment plan. In some embodiments, the second scanning state may be a positioning scanning period.

In some embodiments, the determination module 230 may obtain third scanning data of the subject during the second scanning stage. The third scanning data may be acquired in a third time period in the second scanning stage. A third scanning process of obtaining the third scanning data may not overlap in time with a first scanning process of obtaining the first scanning data and a second scanning process of obtaining the second scanning data or may partially overlap with the first scanning data and/or the second scanning data in time.

In some embodiments, the determination module 230 may determine the target motion state of the subject when the third scanning data is acquired by the MRI device based on the third scanning data and the target correspondence.

In some embodiments, the third scanning data be acquired based on a quick magnetic resonance scan on the subject. In some embodiments, the third scanning data may be obtained during an application phase. More descriptions regarding the application phase may be found in FIG. 1 and the relevant descriptions thereof.

In some embodiments, the third scanning data may be raw data including temporal dimension information. For example, the third scanning data may be a K-space dataset including the temporal dimension information. In some embodiments, the third scanning data may be scanning data that does not include temporal dimension information, that is, scanning data obtained at a particular quick magnetic resonance scan. For example, the third scanning data may be a K-space dataset that does not include the temporal dimension information.

In some embodiments, the third scanning data may correspond to the second FOV. Accordingly, the third scanning data may be scanning data obtained after the quick magnetic resonance scan is performed on the subject in the second FOV.

In some alternative embodiments, the third scanning data may correspond to a third FOV that is different from the second FOV, for example, the third FOV may be smaller than the second FOV, or the third FOV may be larger than the second FOV but smaller than the first FOV, etc.

In some embodiments, the determination module 230 may obtain the third scanning data of the subject by performing the quick magnetic resonance scan on the subject in the second FOV via the MRI device (e.g., the MRI device 110).

In some embodiments, the determination module 230 may determine a K-space dataset in the second scanning data that matches the third scanning data. The K-space dataset in the second scanning data that matches the third scanning data may be referred to as a target K-space dataset.

In some embodiments, the determination module 230 may determine a matching degree between each K-space dataset in the second scanning data and the third scanning data, and determine, based on the matching degree, the K-space dataset in the second scanning data that matches the third scanning data.

The matching degree refers to a parameter configured to measure a similarity between a K-space dataset and the third scanning data. The higher the similarity between the K-space dataset and the third-scanning data is, the higher the matching degree may be.

In some embodiments, the determination module 230 may determine a first data feature of each K-space dataset in the second scanning data, and determine a second data feature of the third scanning data; and determine, based on the first data feature corresponding to each K-space dataset and the second data feature corresponding to the third scanning data, the matching degree between each K-space dataset and the third scanning data.

The data feature is a feature associated with the K-space dataset. In some embodiments, the data feature may include a frequency encoding gradient, a phase encoding gradient, a phase encoding step, a filled trajectory of K-space, or the like, or any combination thereof. In some embodiments, the data feature may include a statistical feature of data in a central region of the K-space, a statistical feature of data in an edge region of the K-space, a statistical feature of data in a specific region of the K-space, a statistical feature of data in an overall region of the K-space, or the like, or any combination thereof.

In some embodiments, the data feature of the data in the central region of the K-space may include a mean, a variance, a standard deviation, etc., of the data in the center region of the K-space. In some embodiments, the data feature of the data in the surrounding region of the K-space may include a mean, a variance, a standard deviation, etc., of the data in the edge region of the K-space. In some embodiments, the data feature of the data in the specific region of the K-space may include a mean, a variance, a standard deviation, etc. of the data in the specific region of the K-space. In some embodiments, the data feature of the data in the overall region of the K-space may include a mean, a variance, a standard deviation, etc. of the data in the overall region of the K-space. The specific region of the K-space may be pre-specified artificially or by the system.

The first data feature refers to a data feature corresponding to a K-space dataset in the second scanning data. The second data feature refers to a data feature corresponding to the third scanning data.

The first data feature and the second data feature may be determined in various ways, and the first data feature and the second data feature may be obtained in a similar way. In some embodiments, the determination module 230 may obtain a feature vector corresponding to the K-space dataset as the first data feature or the second data feature by performing feature extraction on the K-space dataset through a principal components analysis (PCA) algorithm or convolutional neural networks (CNN). The embodiments of the present disclosure do not have a special limitation on the way of extracting the data feature of the K-space dataset, and it is sufficient to adopt the operation known to those skilled in the art.

In some embodiments, the determination module 230 may determine a similarity between the first data feature corresponding to each K-space dataset and the second data feature corresponding to the third scanning data, and determine the similarity as a matching degree between the each K-space dataset and the third scanning data. The manner for determining the similarity may include, but is not limited to, determining a vector distance, etc.

In some embodiments, the determination module 230 may determine the matching degree between the each K-space dataset and the third scanning data based on a matching degree determination model. The matching degree determination model may be a machine learning model. In some embodiments, the matching degree determination model may include various feasible models such as an RNN model, a DNN model, a CNN model, or the like, or any combination thereof.

In some embodiments, an input of the matching degree determination model may include the K-space dataset and the third scanning data, and an output of the matching degree determination model may be the matching degree between the K-space dataset and the third scanning data. In other embodiments, the input of the matching degree determination model may include the third scanning data and each K-space dataset in the second scanning data, and the output of the matching degree determination model may include the matching degree between the each K-space dataset in the second scanning data and the third scanning data.

In some embodiments, the matching degree determination model may be obtained by training samples with labels. In some embodiments, the training sample may include two sample K-space datasets, and the label may be a matching degree between the two sample K-space datasets. In some embodiments, the training sample may be obtained based on historical data, and the label may be determined by systematic or human labeling. For example, the system or human may determine a matching degree between the two sample K-space datasets according to the similarity between the two data features by calculating the data features of the two sample K-space datasets respectively.

In some embodiments, the determination module 230 may determine, based on the matching degree between the each K-space dataset and the third scanning data, one K-space dataset that has a highest matching degree to be a K-space dataset (i.e., the target K-space dataset) that matches the third scanning data.

In some embodiments, the determination module 230 may determine, in the target correspondence, a set of first images in the first scanning data corresponding to the target K-space dataset. The set of first images in the first scanning data corresponding to the target K-space dataset may be referred to as a target image set.

In some embodiments, the determination module 230 may determine a motion state corresponding to the third scanning data based on the target image set. For example, the determination module 230 may determine a motion state of the subject represented by the target image combination to be the target motion state of the subject when the third scanning data is acquired by the MRI device. The target motion state corresponding to the third scanning data may be a position, morphology, etc., corresponding to the subject when the third scanning data is acquired by the imaging device.

In some embodiments, the determination module 230 may determine a three-dimensional imaging result of the subject based on the third scanning data. For example, the determination module 230 may determine the target image set as an image imaging result corresponding to the third scanning data, that is, a reconstruction result corresponding to the third scanning data may be obtained without reconstruction.

It should be noted that although, in the above embodiments, the first scanning data (and the first raw data) being 4D scanning data, the second scanning data (and the second raw data) and the third scanning data being scanning data obtained after the quick magnetic resonance scan are illustrated as examples. The above relevant descriptions are merely provided for the purpose of illustration, and are not intended to limit the scope of the present disclosure. The first scanning data, the second scanning data, and the third scanning data may be other types of scanning data. For example, the first scanning data may be a two-dimensional scanning image sequence including the temporal dimension information, and the second scanning data and the third scanning data may be normal scanning data obtained from the magnetic resonance scan.

The beneficial effects achieved by the embodiments of the present disclosure may at least include: (1) By performing the pre-scanning stage and the application phase, real-time motion state capture of certain organs that are greatly affected by tissue motion such as respiratory motion, heart beating, etc. can be realized, the precision of motion tracking of the target organs and tissues can be improved, and a relatively high real-time requirement can be ensured; (2) By constructing the target correspondence between the K-space dataset and the three-dimensional scanning image based on the scanning result in the pre-scanning stage, the motion state of the motion tissues can be obtained without performing reconstruction on the K-space dataset in the application phase, which effectively reduces the time cost of image reconstruction and the computing power consumption brought by the signal processing and image reconstruction to the computer; (3) By adopting a small FOV imaging in the pre-scanning stage, the count of phase encoding steps can be effectively reduced, which greatly reduces the imaging time consumed by phase encoding, and the severity of image wrap artifacts due to insufficient phase encoding can be fully used to locate the current motion state of the moving tissues; (4) The image wrap may reflect the increase of high-frequency components in the image caused by image overlapping in the K-space dataset, thereby locating the correspondence between different motion states and the change pattern of K-space data; and (5) By obtaining the K-space dataset of the subject in real time in the application phase, and through the K-space dataset obtained in real time and the target correspondence obtained in the preprocessing phase, the motion state may be directly guided and captured using the K-space data, thereby greatly reducing the time taken by the two-dimensional image reconstruction, and rapidly locating the current motion state of the motion tissues, so that the high-speed and real-time three-dimensional imaging of magnetic resonance can be finally realized.

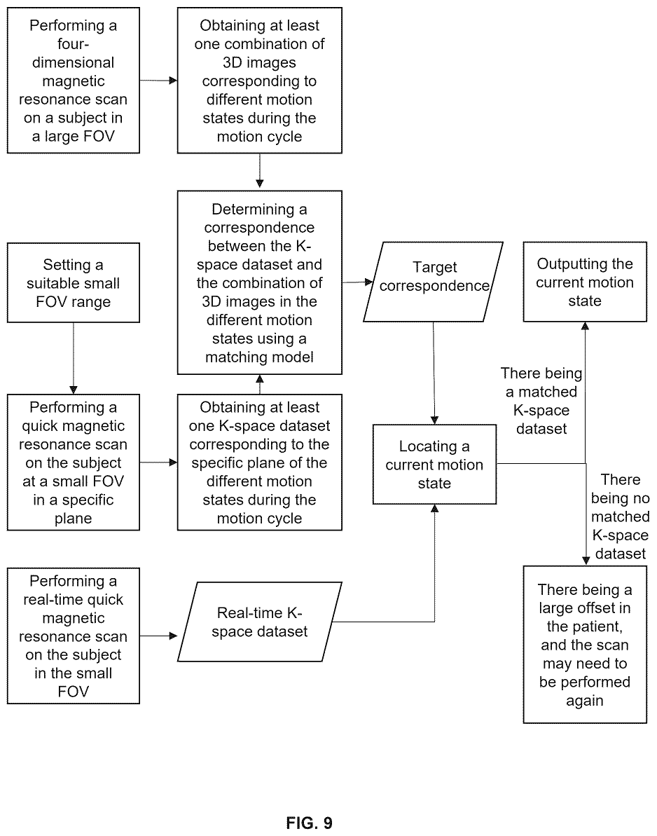

The method for determining a target motion state in some embodiments of the present disclosure in conjunction with the embodiments of FIG. 9 is illustrated below.

The system for determining a target motion state may obtain a target correspondence between first scanning data and second scanning data in each motion state during a motion cycle through a preprocessing phase. The preprocessing phase may include the following operations S11 to S13.

In S11, first raw data may be obtained by performing a four-dimensional magnetic resonance scan on a subject in a large FOV; and at least one set of 3D images corresponding to different motion states during the motion cycle may be obtained based on the first raw data.

In S12, a suitable small FOV range may be set (sizes of a large FOV range and a small FOV range may be relative to each other) and the second raw data may be obtained by performing a quick magnetic resonance scan on the subject at a small FOV in a specific plane; and at least one K-space dataset corresponding to the specific plane of the different motion states during the motion cycle may be obtained based on the second raw data.

It should be noted that the operation S11 and the operation S12 may be performed simultaneously or non-simultaneously.

In S13, the target correspondence may be obtained by determining a correspondence between the K-space dataset and the set of 3D images in the different motion states using a matching model based on the at least one set of 3D images corresponding to the different motion states and the at least one K-space dataset corresponding to the specific plane of different motion states.

The above is the preprocessing phase, and after the preprocessing phase is completed, the system for determining a target motion state may enter an application phase. The application phase may include the following operations S14 to S15.

In S14, a real-time K-space dataset (i.e., the third scanning data) may be obtained by performing a real-time quick magnetic resonance scan on the subject in the small FOV.

In S15, a current motion state may be located based on the real-time K-space dataset and the target correspondence obtained in the preprocessing phase. In the process, if there is a K-space dataset matching the real-time K-space dataset in the target correspondence, the current motion state may be determined according to a set of 3D images corresponding to the matched K-space dataset, and the current motion state may be output. If there is no K-space dataset matching the real-time K-space dataset in the target correspondence, it may be determined that there is a large offset in the patient, and the scan may need to be performed again.

FIG. 10 is a flowchart illustrating an exemplary process for obtaining a target image according to some embodiments of the present disclosure. In some embodiments, the process for obtaining a target image may be performed by the processing device 120 or the system 200 for determining a target motion state. For example, the process 1000 may be stored in a storage device (e.g., the storage device 150) in the form of a program or instruction, and the process 1000 may be implemented when the processing device 120 or the system 200 for determining a target motion state executes the program or instruction. The schematic diagram illustrating the operations of the process 1000 presented below is illustrative. In some embodiments, the process may be accomplished using one or more additional operations not described and/or one or more operations not discussed. Additionally, the order of the operations of the process 1000 illustrated in FIG. 10 and described below is not limiting.

In 1001, first scanning data of a subject during a first scanning stage may be obtained. The first scanning data may correspond to a first FOV. In some embodiments, the operation 1001 may be performed by the obtaining module 210 or the processing device 120.

The operation 1001 is the same as the operation 301. More descriptions may be found above.

In 1002, second scanning data of the subject during the first scanning stage may be obtained. The second scanning data may correspond to a second FOV. In some embodiments, the operation 1002 may be performed by the obtaining module 210.

The operation 1002 is the same as the operation 302. more descriptions may be found above.

In 1003, a target correspondence between the first scanning data and the second scanning data of the subject corresponding to a same motion state during the first scanning stage may be determined based on the first scanning data and the second scanning data. In some embodiments, the operation 1003 may be performed by the analysis module 220.

The operation 1003 is the same as the operation 303. More descriptions may be found above.

In 1004, the target image of the subject during a second scanning stage may be determined based on the target correspondence between the first scanning data and the second scanning data. In some embodiments, the operation 1004 may be performed by the determination module 230.

In some embodiments, the determination module 230 may obtain the third scanning data of the subject during the second scanning stage. More descriptions regarding the obtaining the third scanning data may be found in the relevant descriptions of the operation 304.

In some embodiments, the determination module 230 may determine the target image of the subject during the second scanning stage based on the third scanning data and the target correspondence. In some embodiments, the determination module 230 may determine a K-space dataset (i.e., a target K-space dataset) in the second scanning data that matches the third scanning data, determine a set of 3D images in the first scanning data that corresponds to the target K-space dataset (i.e., a target image combination) in the target correspondence, and determine, based on the target image combination, the target image corresponding to the third scanning data. For example, the determination module 230 may determine any three-dimensional scanning image in the target image combination to be the target image of the subject when the third scanning data is acquired by an imaging device.

More descriptions regarding the determining the target K-space dataset, the target image combination may be found in the relevant descriptions of the operation 304.

In some embodiments of the present disclosure, the K-space dataset of the subject may be obtained in real time in the application phase, and through the K-space dataset obtained in real time and the target correspondence obtained in the preprocessing phase, the motion state may be directly guided and captured using the K-space data, thereby greatly reducing the time consumed by two-dimensional image reconstruction, and quickly locating the current motion state of the motion tissue, so that the high-speed and real-time three-dimensional imaging of the magnetic resonance can be finally realized.

One or more embodiments of the present disclosure provide a device. The device may include at least one processor, and at least one storage. The at least one storage may be configured to store computer instructions; and the at least one processor may be configured to execute at least a portion of the computer instructions to: obtain first scanning data of a subject during a first scanning stage, the first scanning data corresponding to a first FOV; obtain second scanning data of the subject during the first scanning stage, the second scanning data corresponding to a second FOV, and the second FOV being smaller than the first FOV; determine, based on the first scanning data and the second scanning data, a target correspondence between the first scanning data and the second scanning data of the subject in at least one motion state during the first scanning stage; and determine, based on the target correspondence, a target motion state of the subject during a second scanning stage.

One or more embodiments of the present disclosure provide a device. The device may include at least one processor and at least one storage. The at least one storage may be configured to store computer instructions; and the at least one processor may be configured to execute at least a portion of the computer instructions to: obtain first scanning data of a subject during a first scanning stage, the first scanning data corresponding to a first FOV; obtain second scanning data of the subject during the first scanning stage, the second scanning data corresponding to a second FOV, and the second FOV being smaller than the first FOV; determine, based on the first scanning data and the second scanning data, a target correspondence between the first scanning data and the second scanning data of the subject in at least one motion state during the first scanning stage; and determine, based on the target correspondence, a target image of the subject during a second scanning stage.

One or more embodiments of the present disclosure provide a computer-readable storage medium storing computer instructions. When reading the computer instructions in the storage medium, the computer may perform a method including: obtaining first scanning data of a subject during a first scanning stage, the first scanning data corresponding to a first FOV; obtaining second scanning data of the subject during the first scanning stage, the second scanning data corresponding to a second FOV, and the second FOV being smaller than the first FOV; determining, based on the first scanning data and the second scanning data, a target correspondence between the first scanning data and the second scanning data of the subject in at least one motion state during the first scanning stage; and determining, based on the target correspondence, a target motion state of the subject during a second scanning stage.

One or more embodiments of the present disclosure provide a computer-readable storage medium storing computer instructions. When reading the computer instructions in the storage medium, the computer may perform a method including: obtaining first scanning data of a subject during a first scanning stage, the first scanning data corresponding to a first FOV, and the first scanning data including at least one set of first images of the subject in at least one motion state during the first scanning stage; obtaining second scanning data of the subject during the first scanning stage, the second FOV corresponding to a second FOV, and the second FOV being smaller than the first FOV; determining, based on the first scanning data and the second scanning data, a target correspondence between the first scanning data and the second scanning data of the subject in at least one motion state during the first scanning stage; obtaining third scanning data of the subject during a second scanning stage; and determining, based on the target correspondence, a target image of the subject during the second scanning stage.

FIG. 11 is a flowchart illustrating an exemplary process for determining a radiotherapy plan according to some embodiments of the present disclosure. In some embodiments, the process for determining a radiotherapy plan may be performed by the processing device 120 or the system 200 for determining a target motion state. For example, the process 1100 may be stored in a storage device (e.g., the storage device 150) in the form of a program or instruction, and the process 1100 may be implemented when the processing device 120 or the system 200 for determining a target motion state executes the program or instruction. The schematic diagram illustrating the operations of the process 1100 presented below is illustrative. In some embodiments, the process may be accomplished using one or more additional operations not described and/or one or more operations not discussed. Additionally, the order of the operations of the process 1100 illustrated in FIG. 11 and described below is not limiting.

In 1101, first scanning data of a subject during a first scanning stage may be obtained. The first scanning data may correspond to a first FOV. In some embodiments, the operation 1101 may be performed by the obtaining module 210 or the processing device 120.

The operation 1101 is the same as the operation 301. More descriptions may be found above.

In 1102, second scanning data of the subject during the first scanning stage may be obtained. The second scanning data may correspond to a second FOV. In some embodiments, the operation 1102 may be performed by the obtaining module 210.

The operation 1102 is the same as the operation 302. More descriptions may be found above.

In 1103, a target correspondence between the first scanning data and the second scanning data of the subject corresponding to a same motion state during the first scanning stage may be determined based on the first scanning data and the second scanning data. In some embodiments, the operation 1103 may be performed by the analysis module 220.

The operation 1103 is the same as the operation 303. More descriptions may be found above.

In 1104, a target image of the subject during a second scanning stage may be determined based on the target correspondence between the first scanning data and the second scanning data. In some embodiments, the operation 1104 may be performed by the determination module 230.

The operation 1104 is the same as the operation 1004. More descriptions may be found above.

In 1105, a radiotherapy plan of the subject may be determined based on the target image. In some embodiments, the operation 1105 may be performed by the determination module 230.

In some embodiments, the determination module 230 may determine a physiological phase of the subject based on the target image, and determine the radiotherapy plan of the subject based on the physiological phase of the subject in various ways. In some embodiments, the determination module 230 may determine, based on the target image, the physiologic phase of the subject in a way such as image recognition, etc. The embodiments of the present disclosure do not have any special limitation on the way of determining the physiological phase, and it is sufficient to adopt the operation known to those skilled in the art.

In some embodiments, the determination module 230 may determine, based on the physiological phase of the subject, the radiotherapy plan of the subject by querying a preset schedule. In some embodiments, the preset schedule may include a correspondence between a plurality of physiologic phases of a plurality of organ types and a plurality of radiotherapy plans. In some embodiments, the preset schedule may be determined based on prior knowledge or historical data in advance. In some embodiments, the determination module 230 may match a historical radiotherapy plan corresponding to a similar historical physiological phase of one same organ type from historical radiotherapy data based on the physiological phase of the subject, and determine the historical radiotherapy plan as the current radiotherapy plan. The radiotherapy plan may also be determined in other feasible ways, which is not limited herein.

In some embodiments of the present disclosure, the targeted radiotherapy plan may be determined by analyzing the physiological phase of the subject, thereby improving the accuracy of the radiotherapy.

Having thus described the basic concepts, it may be rather apparent to those skilled in the art after reading this detailed disclosure that the foregoing detailed disclosure is intended to be presented by way of example only and is not limiting. Although not explicitly stated here, those skilled in the art may make various modifications, improvements and amendments to the present disclosure. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various parts of this specification are not necessarily all referring to the same embodiment. In addition, some features, structures, or features in the present disclosure of one or more embodiments may be appropriately combined.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that the present disclosure object requires more features than the features mentioned in the claims. Rather, claimed subject matter may lie in less than all features of a single foregoing disclosed embodiment.

In some embodiments, the numbers expressing quantities or properties used to describe and claim certain embodiments of the present disclosure are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

1. A method, comprising:
obtaining first scanning data of a subject during a first scanning phase, the first scanning data corresponding to a first field of view (FOV);
obtaining second scanning data of the subject during the first scanning phase, the second scanning data corresponding to a second FOV, and the second FOV being smaller than the first FOV;
determining, based on the first scanning data and the second scanning data, a target correspondence between the first FOV and the second FOV of the subject in at least one motion state during the first scanning phase; and
determining, based on the target correspondence, a target motion state of the subject during a second scanning phase.

2. The method of claim 1, wherein the determining, based on the target correspondence, a target motion state of the subject during a second scanning phase includes:
obtaining third scanning data of the subject during the second scanning phase; and
determining, based on the third scanning data and the target correspondence, a target motion state of the subject when the third scanning data is acquired by an imaging scanner.

3. The method of claim 2, wherein the determining, based on the third scanning data and the target correspondence, a target motion state of the subject when the third scanning data is acquired by an imaging scanner includes:
determining, based on the third scanning data, a target K-space dataset in the second scanning data matching the third scanning data, wherein the third scanning data corresponds to the second FOV; and
determining the target motion state based on the target correspondence and the target K-space dataset matching the third scanning data.

4. The method of claim 3, wherein the determining, based on the third scanning data, a target K-space dataset in the second scanning data matching the third scanning data includes:
determining a matching degree between each K-space dataset in the second scanning data and the third scanning data; and
determining, based on the matching degree, the target K-space dataset in the second scanning data matching the third scanning data.

5. The method of claim 1, wherein determining the second FOV includes:
determining, based on the first scanning data and a preset condition, the second FOV.

6. The method of claim 5, wherein the preset condition includes:
setting a field of view size of the second FOV to make that an image obtained based on the first scanning data does not include image warp, and an image obtained based on the second scanning data includes image wrap.

7. The method of claim 5, wherein the preset condition includes:
setting a field of view size of the second FOV to make that a temporal resolution of the second scanning data during the first scanning phase satisfies a preset temporal resolution condition and/or image wrap appears in a direction in which motion of the subject has a greatest impact on imaging.

8. The method of claim 1, wherein the second FOV is related to at least one of a scanning parameter, a subject feature, or a resolution requirement.

9. The method of claim 8, wherein determining the second FOV includes:
determining the second FOV through a prediction model based on the at least one of a scanning parameter, a subject feature, or a resolution requirement associated with the second FOV, the prediction model being a neural network model.

10. The method of claim 9, wherein an input of the prediction model further includes the first FOV.

11. The method of claim 9, wherein the input of the prediction model further includes the first scanning data.

12. The method of claim 9, wherein the input of the prediction model further includes a test FOV and test scanning data.

13. The method of claim 1, wherein the first scanning data includes at least one combination of three-dimensional scanning images of the subject in at least one motion state during the first scanning phase, and the obtaining first scanning data of a subject during a first scanning phase includes:
obtaining first raw data of the subject during the first scanning phase, the first raw data corresponding to the first FOV; and
obtaining the at least one combination of three-dimensional scanning images by performing, based on the first raw data, a three-dimensional state segmentation, each combination of the at least one combination of three-dimensional scanning images corresponding to one of the at least one motion state of the subject.

14. The method of claim 13, wherein the second scanning data includes at least one K-space dataset of the subject in the at least one motion state during the first scanning phase, and the obtaining second scanning data of the subject during the first scanning phase includes:
obtaining second raw data of the subject during the first scanning phase, the second raw data corresponding to the second FOV; and
obtaining the at least one K-space dataset by dividing, based on a K-space data variation feature, the second raw data, each set of the at least one K-space dataset corresponding to one of the at least one combination of three-dimensional scanning images.

15. The method of claim 1, wherein the determining, based on the first scanning data and the second scanning data, a target correspondence between the first FOV and the second FOV of the subject in at least one motion state during the first scanning phase includes:
determining, based on the second scanning data, a second correspondence between the second scanning data and at least one motion state during the first scanning phase; and
determining, based on a first correspondence between the first scanning data and at least one motion state during the first scanning phase and the second correspondence, the target correspondence.

16. A method, comprising:
obtaining first scanning data of a subject during a first scanning phase, the first scanning data corresponding to a first FOV;
obtaining second scanning data of the subject during the first scanning phase, the second scanning data corresponding to a second FOV, and the second FOV being smaller than the first FOV;
determining, based on the first scanning data and the second scanning data, a target correspondence between the first FOV and the second FOV of the subject in at least one motion state during the first scanning phase; and
determining, based on the target correspondence, a target image of the subject during a second scanning phase.

17-31. (canceled)

32. A computer-readable storage medium storing computer instructions, wherein when reading the computer instructions in the storage medium, the computer performs a method including:
obtaining first scanning data of a subject during a first scanning phase, the first scanning data corresponding to a first FOV;
obtaining second scanning data of the subject during the first scanning phase, the second scanning data corresponding to a second FOV, and the second FOV being smaller than the first FOV;
determining, based on the first scanning data and the second scanning data, a target correspondence between the first FOV and the second FOV of the subject in at least one motion state during the first scanning phase; and
determining, based on the target correspondence, a target motion state of the subject during a second scanning phase.

33-34. (canceled)

35. The computer-readable storage medium of claim 32 wherein the determining, based on the target correspondence, a target motion state of the subject during a second scanning phase includes:
obtaining third scanning data of the subject during the second scanning phase; and
determining, based on the third scanning data and the target correspondence, a target motion state of the subject when the third scanning data is acquired by an imaging scanner.

36. The computer-readable storage medium of claim 35, wherein the determining, based on the third scanning data and the target correspondence, a target motion state of the subject when the third scanning data is acquired by an imaging scanner includes:
determining, based on the third scanning data, a target K-space dataset in the second scanning data matching the third scanning data, wherein the third scanning data corresponds to the second FOV; and
determining the target motion state based on the target correspondence and the target K-space dataset matching the third scanning data.

37. The computer-readable storage medium of claim 36, wherein the determining, based on the third scanning data, a target K-space dataset in the second scanning data matching the third scanning data includes:
determining a matching degree between each K-space dataset in the second scanning data and the third scanning data; and
determining, based on the matching degree, the target K-space dataset in the second scanning data matching the third scanning data.
